(19) United States
(12) Patent Application Publication
(10) Pub. No.: US 2025/0043316 A1
(43) Pub. Date: Feb. 6, 2025

(54) RECOMBINANT MANUFACTURE OF SANTALENE

(71) Applicant: ISOBIONICS B.V., Geleen (NL)

(72) Inventors: Janani DURAIRAJ, Binningen (CH); Aalt-Jan VAN DIJK, Wageningen (NL); Thamara HESSELINK, Wageningen (NL); Martinus Julius BEEKWILDER, Geleen (NL); Elena MELILLO, Geleen (NL)

(21) Appl. No.: 18/717,450

(22) PCT Filed: Dec. 12, 2022
(86) PCT No.: PCT/EP2022/085351

§ 371 Date: Jun. 7, 2024

(30) Foreign Application Priority Data
Dec. 13, 2021 (EP) 21214164.2

(51) Int. Cl. C12P 5/00; C12N 9/88; C12N 15/70; C12R 1/19

(52) U.S. Cl. CPC C12P 5/007; C12N 9/88; C12N 15/70; C12R 2001/19; C12Y 402/03083

(57) ABSTRACT

The present invention concerns the field of recombinant manufacture of santalene and related products. In particular, it relates to a method for the manufacture of a composition comprising at least one santalene comprising the step of converting farnesyl pyrophosphate into at least one santalene, wherein said conversion is carried out by a polypeptide exhibiting santalene synthase activity. Moreover, the invention contemplates a composition comprising a mixture of beta-santalene and alpha-santalene with an excess of beta-santalene obtainable by the method of the invention. The invention also relates to the use of a santalene synthase polypeptide, a heterologous polynucleotide encoding it, a vector or gene construct comprising said polynucleotide, a host cell or a non-human transgenic organism comprising said gene construct or vector for the manufacture of a composition comprising at least one santalene, preferably, beta-santalene, more preferably a mixture of beta-santalene and alpha-santalene. Further, the present invention relates to a method for manufacturing a composition comprising at least one santalol, preferably beta-santalol, comprising producing a composition comprising at least one santalene by the aforementioned method of the invention and oxidising said at least one santalene, preferably, beta-santalene, into the respective alcohol in order to manufacture a composition comprising at least one santalol, preferably, beta-santalol. Yet, the invention pertains to a kit for the manufacture of a composition comprising at least one santalene comprising the aforementioned polypeptide. heterologous polynucleotide, vector or gene construct, host cell or non-human transgenic organism and to a non-human host cell or non-human transgenic organism expressing a polypeptide exhibiting santalene synthase activity from the aforementioned heterologous polynucleotide. vector or gene construct.

The present invention concerns the field of recombinant manufacture of santalene and related products. In particular, it relates to a method for the manufacture of a composition comprising at least one santalene comprising the step of converting farnesyl pyrophosphate into at least one santalene, wherein said conversion is carried out by a polypeptide exhibiting santalene synthase activity. Moreover, the invention contemplates a composition comprising a mixture of beta-santalene and alpha-santalene with an excess of beta-santalene obtainable by the method of the invention. The invention also relates to the use of a santalene synthase polypeptide, a heterologous polynucleotide encoding it, a vector or gene construct comprising said polynucleotide, a host cell or a non-human transgenic organism comprising said gene construct or vector for the manufacture of a composition comprising at least one santalene, preferably, beta-santalene, more preferably a mixture of beta-santalene and alpha-santalene. Further, the present invention relates to a method for manufacturing a composition comprising at least one santalol, preferably beta-santalol, comprising producing a composition comprising at least one santalene by the aforementioned method of the invention and oxidising said at least one santalene, preferably, beta-santalene, into the respective alcohol in order to manufacture a composition comprising at least one santalol, preferably, beta-santalol. Yet, the invention pertains to a kit for the manufacture of a composition comprising at least one santalene comprising the aforementioned polypeptide, heterologous polynucleotide, vector or gene construct, host cell or non-human transgenic organism and to a non-human host cell or non-human transgenic organism expressing a polypeptide exhibiting santalene synthase activity from the aforementioned heterologous polynucleotide, vector or gene construct.

Sandalwood oil is a major perfumery ingredient. It also serves in many aromatherapeutic applications. Major ingredients of sandalwood oil include alpha santalol and beta santalol. Beta santalol is the major fragrance impact ingredient of sandalwood oil. Sandalwood oil is becoming very scarce, since the sandalwood plant is difficult to cultivate. Beta santalol is made from beta-santalene. In the sandalwood plant, santalol formation is mediated by a sesquiterpene synthase.

Sesquiterpene synthases cyclize the ubiquitous precursor farnesyl pyrophosphate (FPP), by a unique proton transfer cascade. The outcome of the cyclization reaction is determined by the identity of the terpene synthase. Genes encoding plant terpene synthases can be deployed in microbial production of terpenes. Microbial production of santalenes, including alpha-santalene, trans-alpha bergamotene and beta-santalene, has been demonstrated (US2020/0010822 A1), by using a santalene synthase from Cinnamomum camphora, or from Santalum album. However, such microbes produce terpene mixes which consist mostly of alpha-santalene (50%), and only a minor amount of beta-santalene (20%).

Preparations with higher beta-santalene concentrations would be useful, e.g. for imparting sandalwood impressions on fragrance materials. To obtain preparations with higher concentrations of beta-santalene, separation technologies are difficult, since alpha and beta-santalene have very close physicochemical properties. For producing beta-santalene, a terpene synthase which would produce >50% beta-santalene would be needed.

Furthermore, there is a constant need for aroma compositions with novel or improved olfactory properties.

The technical problem underlying the present invention shall be seen as the provision of means and methods complying with the aforementioned needs. The technical problem is solved by the embodiments characterized in the claims and herein below.

The present invention relates to a method for the manufacture of a composition comprising at least one santalene comprising the step of converting farnesyl pyrophosphate into at least one santalene, wherein said conversion is carried out by at least one polypeptide exhibiting santalene synthase activity, wherein said at least one polypeptide comprises

(i) an amino acid sequence selected from the group consisting of:
a) an amino acid sequence as shown in any of SEQ ID NO: 1, 18, or 21 to 28;
b) an amino acid sequence which is at least 60%, at least 70%, at least 80%, at least 85%, at least 90%, at least 95%, at least 98% or at least 99% identical to the amino acid sequences as shown in any of SEQ ID NO: 1, 18, or 21 to 28;
c) an amino acid sequence encoded by a nucleic acid sequence as shown in SEQ ID NO: 2 or 3 or 19;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 2 or 3 or 19; and
e) an amino acid sequence of a fragment of any one of (a) to (d), said fragment encoding a polypeptide exhibiting a santalene synthase activity; or
   (ii) an amino acid sequence selected from the group consisting of:
a) an amino acid sequence as shown in any one of SEQ ID NO: 4 to 17;
b) an amino acid sequence which is at least 60%, at least 70%, at least 80%, at least 85%, at least 90%, at least 95%, at least 98% or at least 99% identical to the amino acid sequences as shown in SEQ ID NO: 4 to 17; and
c) an amino acid sequence of a fragment of (a) or (b), said fragment encoding a polypeptide exhibiting a santalene synthase activity.

The composition manufactured by the method of the invention comprises at least one santalene as defined herein, preferably beta-santalene, or a mixture of both alpha-santalene and beta-santalene. If both alpha-santalene and beta-santalene are present, it is preferred that said composition comprises an excess of beta-santalene over alpha-santalene.

In one preferred embodiment of the method of the invention, said composition comprising at least one santalene, is substantially free of any one or all of the following: cis-alpha-Bergamotene ((AS Nr. 18252-46-5), (E)-beta-Farnesene (CAS Nr. 18794-84-8), trans-beta-Bergamotene (CAS Nr. 15438-94-5) and beta-Bisabolene (CAS Nr.495-61-4). Substantially free means that the composition contains no detectable amounts of cis-alpha-Bergamotene ((AS Nr. 18252-46-5), (E)-beta-Farnesene (CAS Nr. 18794-84-8), trans-beta-Bergamotene (CAS Nr.15438-94-5) and/or beta-Bisabolene (CAS Nr.495-61-4).

In another preferred embodiment of the method of the invention, said composition comprising at least one santalene, in addition comprises Sesquithujene (CAS Nr. 58319 Jun. 5) in detectable amounts. Preferably, detectable amounts of Sesquithujene (CAS Nr. 58319 Jun. 5) means between and including 0.01% and 3% (mol/mol).

In still another preferred embodiment of the method of the invention, said composition comprising at least one santalene, in addition comprises Sesquithujene (CAS Nr. 58319 Jun. 5) in detectable amounts, and the composition is substantially free of any one or all of the following: cis-alpha-Bergamotene ((AS Nr. 18252-46-5), (E)-beta-Farnesene (CAS Nr. 18794-84-8), trans-beta-Bergamotene (CAS Nr.15438-94-5) and beta-Bisabolene (CAS Nr.495-61-4). “%” in the context of the concentration of a solution as referred to herein means percentage (mol/mol) if not indicated otherwise.

It is to be understood that in the specification and in the claims, “a” or “an” can mean one or more of the items referred to in the following depending upon the context in which it is used.

Thus, for example, reference to “an” item can mean that at least one of said item can be utilized.

As used in the following, the terms “have”, “comprise” or “include” are meant to have either a non-limiting meaning or a limiting meaning. Thus, having a limiting meaning these terms may refer to a situation in which, besides the feature introduced by these terms, no other features are present in an embodiment described, i.e. the terms have a limiting meaning in the sense of “consisting of” or “essentially consisting of”. Having a non-limiting meaning, the terms refer to a situation where besides the feature introduced by these terms, one or more other features are present in an embodiment described.

Further, as used in the following, the terms “preferably”, “more preferably”, “most preferably”, “particularly”, “more particularly”, “typically”, and “more typically” are used in conjunction with features in order to indicate that these features are preferred features, i.e. the terms shall indicate that alternative features may also be envisaged in accordance with the invention.

Further, it will be understood that the term “at least one” as used herein means that one or more of the items referred to following the term may be used in accordance with the invention. For example, if the term indicates that at least one item shall be used this may be understood as one item or more than one item, i.e. two, three, four, five or any other number. Depending on the item the term refers to the skilled person understands as to what upper limit the term may refer, if any.

The method according to the present invention may either consist of the aforementioned step or may comprise additional steps. Such additional steps may be steps of pre-treatments or steps required for the manufacture of a composition comprising at least one santalene such as purification steps.

The term “manufacture” as used herein refers to the generation of a composition comprising at least one santalene, in particular, a composition comprising beta-santalene, more preferably, a composition comprising a mixture of alpha-santalene and beta-santalene, most preferably, with an excess of beta-santalene. The manufacture may yield any degree of purity of the said at least one santalene in the composition. The higher the degree of envisaged purity, the more additional purification will be required. The method may be carried out ex-vivo, e.g., in one or more reaction vials. Alternatively, the method may be carried out entirely or in part in an organism such as a microorganism including the host cells referred to herein elsewhere or a non-human transgenic organism, preferably a non-vertebrate transgenic organism, including plants or microorganisms.

The term “santalene” as used in accordance with the present invention relates to a tri-cyclic sequiterpene selected from the group consisting of alpha-santalene (CAS number 512-61-8; 6,7-Dimethyl-7-(4-methylpent-3-enyl)-2,3,4,5-tetrahydro-1H-tricyclo [2.2.1.02.6] heptan; molecular formula C₁₅H₂₄), beta-santalene (CAS number 511-59-1, (1R,3R,4S)-3-Methyl-2-methyliden-3-(4-methylpent-3-enyl) bicycle [2.2.1] heptan; molecular formula C₁₅H₂₄), and epi-beta-santalene (CAS number 25532-78-9; (3S)-3-Methyl-2-methyliden-3-(4-methylpent-3-enyl) bicycle [2.2.1] heptan; molecular formula C15H24) and trans-a-bergamotene (CAS number 13474-59-4; herein after also referred to as trans-alpha bergamotene or bergamotene in short). Preferably, said santalene is beta-santalene (CAS number 511-59-1, molecular formula C15H24).

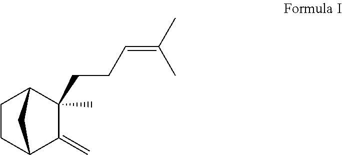

Formula I is a representation of (-)-b-santalene (CAS number 511-59-1; hereinafter referred to as beta-santalene)

The “at least one santalene” referred to in accordance with the present invention is, preferably, beta-santalene. More preferably, it is a mixture of beta-santalene and alpha-santalene with an excess of beta-santalene. Most preferably, said beta-santalene is present in the composition in a relative amount to total santalenes of at least about 50%, at least about 60%, at least about 70%, preferably, between about 50% and about 80%, between about 60% and about 75%, between about 65% and about 70%, more preferably at least about 67%. Total santalenes as referred to herein encompass all santalenes found in the composition. Preferably, these are alpha-santalene, beta-santalene, and bergamotene. Other minor santalenes may occur as well.

Further preferably, the ratio of bergamotene to beta-santalene is below 1.0, for example equal to or below 0.9, 0.8 or 0.7, for example equal to or below 0.5, 0.4, 0.3, 0.2 or even 0.1. In another aspect of the invention, the total santalenes comprise only 10% or less bergamotene and an excess of beta santalene over alpha santalene. In a further aspect of the invention, the percentage (mol/mol) of alpha santalene and bergamotene together is less than the percentage (mol/mol) of beta santalene in the total of santalenes.

In a further aspect of the invention, the composition comprises at least one santalene, preferably an excess of beta-santalene over alpha-santalene, wherein the composition, in addition a) comprises Sesquithujene (CAS Nr. 58319 Jun. 5) in detectable amounts, preferably between and including 0.01% and 3% (mol/mol); b) is substantially free of any or all of the following; cis-alpha-Bergamotene ((AS Nr. 18252-46-5), (E)-beta-Farnesene (beta-farnesene, CAS Nr. 18794-84-8), trans-beta-Bergamotene (CAS Nr.15438-94-5) and beta-Bisabolene (CAS Nr.495-61-4), or a combination of a) and b).

The absence or at least largely reduced amounts of (E)-beta-Farnesene is useful for example if a composition comprising a santalene is used to produce santalol, as in such oxidation reactions the presence of (E)-beta-Farnesene is less desirable.

The term “polypeptide” as used in accordance with the present invention refers to contiguous sequence of amino acid linked to each other by peptide bounds. A polypeptide according to the invention, typically, comprises at least 50, at least 100 or at least 200 amino acids in length such that the amino acid chain may form a three-dimensional structure required to exert the enzymatic activity or enzymatic activities referred to elsewhere herein. The term “protein” may be used interchangeably herein.

The term “santalene synthase activity” as used to herein refers to an activity of the enzyme that allows for converting farnesyl pyrophosphate as a starting material into at least one santalene. Typically, santalene synthases are capable of catalysing a mixture of sesquiterpenoids from (2E,6E)-farnesyl diphosphate, such as alpha-santalene, beta-santalene, epi-beta-santalene and trans-alpha bergamotene, as well as traces of alpha-farnesene and beta-farnesene. They may also use (Z,Z)-farnesyl diphosphate isomer for conversion into alpha-endo-bergamotene, alpha-santalene, (Z)-beta-farnesene, epi-beta-santalene, and beta-santalene (E.C. 4.2.3.81).

Preferably, the polypeptide having santalene synthase activity in accordance with the present invention comprises an amino acid sequence selected from the group consisting of:
a) an amino acid sequence as shown in any of SEQ ID NO: 1, 18, or 21 to 28;
b) an amino acid sequence which is at least 60%, at least 70%, at least 80%, at least 85%, at least 90%, at least 95%, at least 98% or at least 99% identical to the amino acid sequences as shown in any of SEQ ID NO: 1, 18, or 21 to 28;
c) an amino acid sequence encoded by a nucleic acid sequence as shown in SEQ ID NO: 2 or 3 or 19;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 2 or 3 or 19; and
e) an amino acid sequence of a fragment of any one of (a) to (d), said fragment encoding a polypeptide exhibiting a santalene synthase activity.

In one aspect of the invention, the polypeptide is a dual function synthase, i.e. it is a santalene synthase, yet is also Sesquithujene synthase (EC 4.2.3.102 (2E,6E)-farnesyl-diphosphate diphosphate-lyase (sesquithujene-forming)).

In a further aspect of the invention, the polypeptide of the invention is not producing beta-farnesene in substantial amounts when the santalenes and/or sesquithujene are produced.

Preferably, the polypeptide having santalene synthase activity is from or based on a polypeptide from the genus Oryza, for example, but not limited to, from O. meridionalis, O. glumipatula, O. sativa, O. rufipogon, O. glaberrima, O. nivara, O. barthii, O. punctata.

The polypeptide having santalene synthase activity in accordance with the present invention is preferably producing beta-santalene and alpha-santalene in a ratio that is equal to or greater than 1, preferably at least 1.1 and more preferably at least 1.2 and even more preferably 1.3, under conditions suitable for the production of these santalenes.

These santalene synthases of the invention produce beta-santalene and alpha-santalene, preferably measured by GC-FID, in a molar ratio of beta-santalene to alpha-santalene that is equal to or greater than 1; for example, the ratio is at least 1.05, 1.1, 1.2, 1.3, 1.4, 1.5, 1.6, 1.7, 1.8, 1.9 or at least 2. The ratio of beta-santalene to alpha-santalene may be at least 3:1, preferably at least 4:1, more preferably at least 5:1, even more preferably 6:1, yet even more preferably at least 7:1, most preferably at least 8:1 and even at least 9:1. In one aspect of the invention, the ratio is not greater than 100:1.

Furthermore, another aspect of the invention relates to polypeptide having santalene synthase activity as defined herein that produce beta santalene in excess of alpha santalene which is in excess of trans-alpha bergamotene. Typically, santalene synthases known in the art produce sizeable amounts of bergamotene, which for some applications is an undesired santalene. Surprisingly, the polypeptide having santalene synthase activity of the invention showed to produce only small amounts of bergamotene (less than 10% of total santalenes) and produced beta santalene in excess of alpha-santalene

The “sequence identity” referred to herein above defines a relationship between amino acid sequences or nucleic acid sequences and can be determined by comparing those sequences. Usually, sequence identities are determined by comparing two sequences over the whole length of the sequences but may also be compared only for a part of the sequences aligning with each other. Preferably, the sequence identities are compared over the whole length of the sequences, herein. Sequence identity refers to the degree of relatedness between polypeptide sequences or nucleic acid sequences. It will be expressed in the percentage of identical amino acids or nucleotides in two sequences compared to each other. Accordingly, upon aligning two sequences, the number of matching amino acids or nucleotides between those sequences is, in general, determined and put into relation to the total number of amino acids or nucleotides in the aligned sequence or sequence part. For instance, variant sequences may be defined by their sequence identity when compared to a parent sequence, i.e. an amino acid sequence as shown in any one of SEQ ID NO: 1, 18, or 21 to 28, or a nucleic acid sequence as shown in SEQ ID NO: 2 or 3 or 19. To determine the percent-identity between two sequences in a first step a pairwise sequence alignment is generated between those two sequences, wherein the two sequences are aligned over their complete, entire or full length (i.e., a pairwise global alignment). The alignment is generated with a program or software described herein. The preferred alignment for the purpose of this invention is that alignment, from which the highest sequence identity can be determined.

Sequence alignments can be generated with a number of software tools, such as Needleman and Wunsch algorithm-Needleman, Saul B. & Wunsch, Christian D. (1970). “A general method applicable to the search for similarities in the amino acid sequence of two proteins”. Journal of Molecular Biology 48 (3): 443-453. This algorithm is, for example, implemented into the “NEEDLE” program, which performs a global alignment of two sequences. The NEEDLE program, is contained within, for example, the European Molecular Biology Open Software Suite (EMBOSS). EMBOSS—a collection of various programs: The European Molecular Biology Open Software Suite (EMBOSS), Trends in Genetics 16 (6), 276 (2000). BLOSUM (BLOcks SUbstitution Matrix)-typically generated on the basis of alignments of conserved regions, e.g., of protein domains (Henikoff S, Henikoff JG: Amino acid substitution matrices from protein blocks. Proceedings of the National Academy of Sciences of the USA. 1992 Nov. 15; 89 (22):

10915-9). One out of the many BLOSUMs is “BLOSUM62”, which is often the “default” setting for many programs, when aligning protein sequences. BLAST (Basic Local Alignment Search Tool)-consists of several individual programs (BlastP, BlastN) which are mainly used to search for similar sequence in large sequence databases. BLAST programs also create local alignments. Typically used is the “BLAST” interface provided by NCBI (National Centre for Biotechnology Information), which is the improved version (“BLAST2”). The “original” BLAST: Altschul, S. F., Gish, W., Miller, W., Myers, E .W. & Lipman, D. J. (1990) “Basic local alignment search tool.” J. Mol. Biol. 215:403-410; BLAST2: Altschul, Stephen F., Thomas L. Madden, Alejandro A. Schaffer, Jinghui Zhang, Zheng Zhang, Webb Miller, and David J. Lipman (1997),

“Gapped BLAST and PSI-BLAST: a new generation of protein database search programs”, Nucleic Acids Res. 25:3389-3402.

Sequence identity as used herein is, preferably, the value as determined by the EMBOSS Pairwise Alignment Algorithm “Needle”. In particular, the NEEDLE program from the EMBOSS package can be used (version 2.8.0 or higher, EMBOSS: The European Molecular Biology Open Software Suite—Rice, P., et al. Trends in Genetics (2000) 16:276-277;http://emboss.bioinformatics.nl) using the NOBRIEF option (‘Brief identity and similarity’ to NO) which calculates the “longest-identity”. The identity between the two aligned sequences is calculated in such a case as follows: Number of corresponding positions in the alignment showing an identical amino acid in both sequences divided by the total length of the alignment after subtraction of the total number of gaps in the alignment. For alignment of amino acid sequences the default parameters are: Matrix=Blosum62; Open Gap Penalty=10.0; Gap Extension Penalty=0.5. For alignment of nucleic acid sequences the default parameters are: Matrix=DNAfull; Open Gap Penalty=10.0; Gap Extension Penalty=0.5.

Variant amino acid or nucleic acid sequences as referred to herein may be naturally occurring variations such as allelic variants or orthologous, paralogous or homologous variants. Alternatively, such sequences may be artificially generated, e.g., in an attempt to improve a property of the enzyme or nucleic acid (e.g., improved expression of the enzyme or increased enzymatic activity of the enzyme) by a biological technique known to the skilled person in the art, such as, e.g., molecular evolution or rational design, or by using a mutagenesis technique known in the art and described elsewhere herein (random mutagenesis, site-directed mutagenesis, directed evolution, gene recombination, etc.). Typically, variants of the polypeptides with santalene synthase activity according to the invention are polypeptides with one or several amino acid substitutions compared to the amino acid sequence of any of SEQ ID NO: 1, 18, or 21 to 28, preferably, artificial amino acid sequences.

Variant nucleic acid sequences encoding an amino acid sequence encoded by a nucleic acid sequence as shown in SEQ ID NO: 2 or 3 or 19 or an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 2 or 3 or 19 may differ from the nucleic acid sequences shown in SEQ ID NO: 2or 3 or 19 for reasons set forth elsewhere herein due to at least one nucleotide substitution, addition and/or deletion. It will be understood that polynucleotides comprising such variant nucleic acid sequences as referred to herein, preferably, are capable of hybridizing to each other under stringent hybridization conditions. Stringent hybridization conditions as referred to herein are, preferably, 6 x sodium chloride/sodium citrate (SSC) at approximately 45° C., followed by one or more wash steps in 0.2 x SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1 to 5 x SSC (pH 7.2). If organic solvent is present in the abovementioned buffer, for example 50% formamide, the temperature under standard conditions is approximately 42° C. The hybridization conditions for DNA: DNA hybrids are, preferably, 0.1 x SSC and 20°° C. to 45° C., preferably between 30°° C. and 45° C. The hybridization conditions for DNA: RNA hybrids are, preferably, 0.1 x SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Thus, variant nucleic acid sequences can be derived from polynucleotides which are capable of hybridizing under stringent hybridization conditions to nucleic acid sequences encoding an amino acid sequence encoded by a nucleic acid sequence as shown in SEQ ID NO: 2 or 3 or 19 or an amino acid sequence encoded by a nucleic acid sequence which is at least 60%, at least 70%, at least 80%, at least 85%, at least 90%, at least 95%, at least 98% or at least 99% identical to the nucleic acid sequence as shown in SEQ ID NO: 2 or 3 or 19.

The invention further relates to a method for preparing a variant polypeptide having a santalene synthase activity, comprising the steps of:
(a) selecting a nucleic acid according to any one of SEQ ID NO: 2 or 3 or 19;
(b) modifying the selected nucleic acid to obtain at least one mutant nucleic acid;
(c) transforming host cells or unicellular organisms as defined herein with the mutant nucleic acid sequence to express a polypeptide encoded by the mutant nucleic acid sequence;
(d) screening the polypeptide for at least one modified property; and,
(e) optionally, if the polypeptide has no desired variant santalene synthase activity, repeating the process steps (a) to (d) until a polypeptide with a desired variant santalene synthase activity as defined herein is obtained;
(f) optionally, if a polypeptide having a desired variant santalene synthase activity was identified in step (d), isolating the corresponding mutant nucleic acid obtained in step (c).

A fragment as referred to above may be a polypeptide consisting of any amino acid sequence of the above-mentioned sequences and sequence variants that is of sufficient length of exhibiting a santalene synthase activity specified above. It is, thus, preferably envisaged that a fragment having the aforementioned biological activity of the polypeptide comprises the amino acid sequence of the catalytically active region of a santalene synthase. Typically, a fragment consists of at least 20, at least 30, at least 40, at least 50, at least 100, at least 150 or at least 200 contiguous amino acids in length from the above-mentioned sequences or sequence variants.

Preferably, a fragment of a polypeptide as referred to herein or a variant polypeptide as referred to herein comprises at least one, preferably, at least two, more preferably, three Pfam domains. Typically, the Pfam domains referred to in accordance with the present invention are the N-terminal domain of terpene synthase (PF01397.21), the metal binding domain of terpene synthase (PF03936.16) and Pfam domain of Trichodiene synthase TRI5 (PF06330.11). Pfam domains as referred to herein are to be analysed using version 32.0 of PFAM, for PFAM details see “The Pfam protein families data-base in 2019: S. El-Gebali, J. Mistry, A. Bateman, S. R. Eddy, A. Luciani, S. C. Potter, M. Qureshi, L. J. Richardson, G. A. Salazar, A. Smart, E. L. L. Sonnhammer, L. Hirsh, L. Paladin, D. Piovesan, S. C. E. Tosatto, R. D. Finn Nucleic Acids Research (2019) and http://pfam.xfam.org/.

Preferably, the polypeptides or fragments having santalene synthase activity according to the present invention shall comprise at least one, preferably all, of the conserved domains shown in FIG. 4. Conserved domains are indicated in FIG. 4 by light letters on black background. Preferably, conserved domains shall consist of at least 3, at least 4, at least 5, at least 6, at least 7, at least 8, at least 9, at least 10, at least 15 or at least 20 amino acids. Particular preferred conserved domains referred to in accordance with the present invention are those having the following amino acids of SEQ ID NO: 1: amino acids 82 to 96, 133 to 146, 148 to 156, 163 to 185, 195 to 205, 214 to 225, 227 to 233, 235 to 242, 345 to 352, 375 to 382, 400 to 409 and 411 to 419.

In another embodiment, the santalene synthase of the invention comprises in its N-Terminal region a GRXCX4W motif (SEQ ID NO: 20), characterized in that the amino acid Glycine is followed by Arginine followed by any amino acid, followed by a Cysteine followed by four amino acids of any type, and finally a Tryptophan.

In a preferred embodiment, the santalene synthase of the invention or a fragment thereof has an amino acid stretch corresponding to the amino acid positions 281 to 341 of SEQ ID NO: 1 with no more than 10, 9, 8, 7, 6, 5, 4, 3, 2, 1, or 0 changes compared to said amino acid stretch in SEQ ID NO: 1. In a further aspect of the invention, changes in said amino acid stretch corresponding to positions 281 to 341 of SEQ ID NO: 1 are in any of those amino acids corresponding to positions 293 to 295 and/or 317 of SEQ ID NO: 1.

Preferably, the polypeptide having santalene synthase activity in accordance with the present invention may yet comprise and amino acid sequence selected from the group consisting of:
a) an amino acid sequence as shown in any one of SEQ ID NO: 4 to 17;
b) an amino acid sequence which is at least 60%, at least 70%, at least 80%, at least 85%, at least 90%, at least 95%, at least 98% or at least 99% identical to the amino acid sequences as shown in SEQ ID NO: 4 to 17;
c) an amino acid sequence of a fragment of (a) or (b), said fragment encoding a polypeptide exhibiting a santalene synthase activity.

The aforementioned polypeptide exhibiting santalene synthase activity may also be comprised in a fusion polypeptide. Such a fusion polypeptide comprises in addition to the amino acid sequence of the polypeptide exhibiting santalene synthase activity one or more additional amino acid sequences. Said additional amino acid sequences may be polypeptides having other enzymatic activities, such as farnesyl pyrophosphate synthases or cytochrome P450 monooxygenases as specified elsewhere herein or polypeptides or peptides having marker or label functions for, e.g., monitoring proper expression or for purification purposes, such as tags (e.g., MYC tag, FLAG tag, His tag, etc.) or fluorescent proteins (e.g., GFP, BFP, YFP or CFP).

Further, the present disclosure is directed to a method for preparing santalenes and santalols, the method comprising converting farnesyl diphosphate (FPP) santalene in the presence of an enzyme, the enzyme comprising a first segment comprising a tag peptide and a second segment comprising a santalene synthase according to the invention. An enzyme comprising said first and said second segment may herein be referred to as a ‘tagged enzyme’.

The tag peptide is preferably selected from the group of nitrogen utilization proteins (NusA), thioredoxins (Trx), maltose-binding proteins (MBP), Glutathione S-transferases (GST), Small Ubiquitin-like Modifier (SUMO) or Calcium-binding proteins (Fh8), and functional homologues thereof. As used herein, a functional homologue of a tag peptide is a tag peptide having at least about the same effect on the solubility of the tagged enzyme, compared to the non-tagged enzyme. Typically, the homologue differs in that one or more amino acid residues have been inserted, substituted, deleted from, or extended to the peptide of which it is a homologue. The homologue may in particular comprise one or more substitutions of a hydrophilic amino acid for another hydrophilic amino acid, or of a hydrophobic amino acid for another. The homologue may, in particular, have a sequence identity of at least 40%, more in particular of at least 50%, preferably of at least 55%, more preferably of at least 60%, at least 70%, at least 75%, at least 80%, at least 85%, at least 90%, at least 95%, at least 98%, or at least 99% sequence identity with the sequence of a NusA, Trx, MBP, GST, SUMO or Fh8.

Particularly suitable is maltose-binding protein from Escherichia coli, or a functional homologue thereof.

The use of a tagged enzyme according to the invention is in particular advantageous in that it may contribute to an increased production, especially increased cellular production of a terpenoid or a terpene, such as santalene and/or sesquithujene.

For improved solubility of the tagged enzyme (compared to the enzyme without the tag), the first segment of the enzyme is preferably bound at its C-terminus to the N-terminus of the second segment. Alternatively, the first segment of the tagged enzyme is bound at its N-terminus to the C-terminus of the second segment.

Further, the present invention is directed to a nucleic acid comprising a nucleotide sequence encoding a polypeptide, the polypeptide comprising a first segment comprising a tag peptide, preferably an MBP, a NusA, a Trx, a GST, a SUMO or anFh8-tag, or a functional homologue of any of these, and a second segment comprising a santalene synthase. The second segment may, for instance, comprise an amino acid sequence as shown in any one of SEQ ID NO: 1, 4 to 17, or a functional analogue thereof. One example is the synthetic fusion protein of SEQ ID NO: 18.

Further, the present invention is directed to a host cell comprising said nucleic acid encoding said tagged santalene synthase.

A specific nucleic acid according to the invention encoding a tagged enzyme is shown in SEQ ID NO: 19. The host cell may in particular comprise a gene comprising any of these sequences, or a functional analogue thereof.

In the method of the present invention, farnesyl pyrophosphate is enzymatically converted into at least one santalene by a santalene synthase as specified herein.

The aforementioned conversion step may be carried out in vitro, i.e. in a suitable reaction vial containing all components required for the conversion as described above. The skilled person is well aware of how to adjust the reaction conditions such that the reaction will be carried out efficiently. For example, suitable buffers may be used to provide the components in an environment having a suitable pH and suitable salt concentrations. A suitable temperature in such a setting can be applied as well without further ado.

Alternatively, the conversion step may be carried out in a host cell as described elsewhere herein. Preferably, the host cell is selected from the group consisting of: a bacterial cell, a yeast cell, a fungal cell, an algal cell or a cyanobacterial cell, a non-human animal cell or a non-human mammalian cell, and a plant cell. It is to be understood that the host cell shall be capable of producing santalene. If necessary, the host cell needs to be genetically modified in order to express enzymes or proteins required for the santalene synthesis including the aforementioned santalene synthase. The host cell shall be cultivated under conditions and for a time sufficient to allow expression of the aforementioned enzymes and for conversion of farnesyl pyrophosphate into at least one santalene. Particular preferred conditions are also described in the accompanying Examples, below, or known to those skilled in the art.

Yet, the conversion step of the method of the present invention may also be carried out in an organism, typically a multi-cellular organism such as the transgenic non-human organism referred to elsewhere herein. Typically, said organism is genetically modified such that the enzymes required for conversion of farnesyl pyrophosphate into at least one santalene are expressed. The skilled person is, however, well aware of what conditions need to be applied depending on the choice of a given non-human transgenic organism.

In case, the method of the invention is carried out in vivo, i.e. in a host cell or a non-human transgenic organism it will be understood that the said host cell or non-human transgenic organism shall express the polypeptide exhibiting santalene synthase activity as specified above such that the conversion of farnesyl pyrophosphate into at least one santalene can be carried out in said host cell or non-human transgenic organism. Preferably, said polypeptide exhibiting santalene synthase activity is encoded by a heterologous polynucleotide, a vector or a gene construct.

The term “heterologous polypeptide” in this context means that the polynucleotide encoding the polypeptide exhibiting santalene synthase activity is not naturally occurring in the host cell or organism into which it is introduced. Thus, a heterologous polynucleotide originates from a first species or is an artificially modified polynucleotide, while the host cell or non-human transgenic organism is from a second species that differs from said first species. A heterologous polynucleotide may be comprised in a vector or gene construct as specified herein below. Alternatively, it may be introduced into the genome of a host cell or non-human transgenic organism such that upon integration into the genome the polypeptide exhibiting santalene synthase activity encoded by said heterologous polynucleotide will be expressed. Typically, the heterologous polynucleotide shall be integrated into the genome of the host cell or non-human transgenic organism at a locus that allows expression of the heterologous polynucleotide, e.g., in proximity to an endogenous promoter.

The term “vector”, preferably, encompasses phage, plasmid, cosmids, viral vectors as well as artificial chromosomes, such as bacterial or yeast artificial chromosomes (YAC). The vector encompassing the polynucleotide of the present invention, preferably, further comprises selectable markers for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, f-mating, natural competence, carbon-based clusters, chemically mediated transfer, electroporation or particle bombardment. Suitable methods for the transformation or transfection of host cells, including plant cells, can be found in Sambrook et al. (loc. cit.) and other laboratory manuals, such as Methods in Molecular Biology, 1995, Vol. 44, Agrobacterium protocols, Ed.: Gartland and Davey, Humana Press, Totowa, New Jersey. Alternatively, a plasmid vector may be introduced by heat shock or electroporation techniques. Should the vector be a virus, it may be packaged in vitro using an appropriate packaging cell line prior to application to host cells.

Preferably, the vector referred to herein is suitable as a cloning vector, i.e. replicable in microbial systems. Such vectors ensure efficient cloning in bacteria and, preferably, yeasts or fungi and make possible the stable transformation of plants. Those which must be mentioned are, in particular, various binary and co-integrated vector systems which are suitable for the T DNA-mediated transformation. Such vector systems are, as a rule, characterized in that they contain at least the vir genes, which are required for the Agrobacterium-mediated transformation, and the sequences which delimit the T-DNA (T-DNA border). These vector systems, preferably, also comprise further cis-regulatory regions such as promoters and terminators and/or selection markers with which suitable transformed host cells or organisms can be identified. While co-integrated vector systems have vir genes and T DNA sequences arranged on the same vector, binary systems are based on at least two vectors, one of which bears vir genes, but no T-DNA, while a second one bears T DNA, but no vir gene. As a consequence, the last-mentioned vectors are relatively small, easy to manipulate and can be replicated both in E. coli and in Agrobacterium. These binary vectors include vectors from the pBIB-HYG, pPZP, pBecks, pGreen series. Preferably used in accordance with the invention are Bin19, pBI101, pBinAR, pGPTV and pCAMBIA. An overview of binary vectors and their use can be found in Hellens et al, Trends in Plant Science (2000) 5, 446-451. Furthermore, by using appropriate cloning vectors, the polynucleotides can be introduced into host cells or organisms such as plants or animals and, thus, be used in the transformation of plants, such as those which are published, and cited, in: Plant Molecular Biology and Biotechnology (CRC Press, Boca Raton, Florida), chapter 6/7, pp. 71-119 (1993); F. F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus 1991, Annu. Rev. Plant Physiol. Plant Molec. Biol. 42, 205 225.

More preferably, the vector of the present invention is an expression vector. In such an expression vector, i.e. a vector which comprises the polynucleotide of the invention having the nucleic acid sequence operatively linked to an expression control sequence (also called “expression cassette”) allowing expression in prokaryotic or eukaryotic cells or isolated fractions thereof. Suitable expression vectors are known in the art such as Okayama-Berg cDNA expression vector pcDV1 (Pharmacia), pCDM8, pRc/CMV, pcDNA1, pcDNA3 (Invitrogen) or pSPORT1 (GIBCO BRL). Further examples of typical fusion expression vectors are pGEX (Pharmacia Biotech Inc; Smith 1988, Gene 67:31-40), pMAL (New England Biolabs, Beverly, MA) and pRIT5 (Pharmacia, Piscataway, NJ), where glutathione S transferase (GST), maltose E-binding protein and protein A, respectively, are fused with the recombinant target protein. Examples of suitable inducible nonfusion E. coli expression vectors are, inter alia, pTrc (Amann 1988, Gene 69:301-315) and pET 11d (Studier 1990, Methods in Enzymology 185, 60-89). The tar-get gene expression of the pTrc vector is based on the transcription from a hybrid trp-lac fusion promoter by host RNA polymerase. The target gene expression from the pET 11d vector is based on the transcription of a T7-gn10-lac fusion promoter, which is mediated by a co-expressed viral RNA polymerase (T7 gn1). This viral polymerase is provided by the host strains BL21 (DE3) or HMS174 (DE3) from a resident lambda-prophage which harbours a T7 gn1 gene under the transcriptional control of the lacUV 5 promoter. The skilled worker is familiar with other vectors which are suitable in prokaryotic organisms; these vectors are, for example, in E. coli, pLG338, pACYC184, the pBR series such as pBR322, the pUC series such as pUC18 or pUC19, the M113mp series, pKC30, pRep4, pHS1, pHS2, pPLc236, pMBL24, pLG200,pUR290, pIN-III113-B1, lambdagt11 or pBdCI, in Streptomyces pIJ101, plJ364, plJ702 or pIJ361, in Bacillus pUB110, pC194 or pBD214, in Corynebacterium pSA77 or pAJ667.Examples of vectors for expression in the yeast S. cerevisiae comprise pYep Sec1 (Baldari 1987, Embo J. 6:229-234), pMFa (Kurjan 1982, Cell 30:933-943), pJRY88 (Schultz 1987, Gene 54:113-123) and pYES2 (Invitrogen Corporation, San Diego, CA). Vectors and pro-cesses for the construction of vectors which are suitable for use in other fungi, such as the filamentous fungi, comprise those which are described in detail in: van den Hondel, C. A. M. J. J., & Punt, P. J. (1991) “Gene transfer systems and vector development for filamentous fungi, in: Applied Molecular Genetics of fungi, J. F. Peberdy et al., Ed., pp. 1-28, Cambridge University Press: Cambridge, or in: More Gene Manipulations in Fungi (J. W. Bennett & L. L. Lasure, Ed., pp. 396-428: Academic Press: San Diego). Further suitable yeast vectors are, for example, pAG-1, YEp6, YEp13 or pEMBLYe23. As an alternative, the polynucleotides of the present invention can be also expressed in insect cells using baculovirus expression vectors. Baculovirus vectors which are available for the expression of proteins in cultured insect cells (for example Sf9 cells) comprise the pAc series (Smith 1983, Mol. Cell Biol. 3:2156-2165) and the pVL series (Lucklow 1989, Virology 170:31-39).

Yet the vector may be an integration vector. An integration vector refers to a DNA molecule, linear or circular, that can be incorporated, e.g., into a microorganism's genome, such as a bacteria's genome, and provides for stable inheritance of a gene encoding a polypeptide of interest, such as the santalene synthase of the invention. The integration vector generally comprises one or more segments comprising a gene sequence encoding a polypeptide of interest under the control of (i.e., operably linked to) additional nucleic acid segments that provide for its transcription.

Such additional segments may include promoter and terminator sequences, and one or more segments that drive the incorporation of the gene of interest into the genome of the target cell, usually by the process of homologous recombination. Typically, the integration vector will be one which can be transferred into the target cell, but which has a replicon which is non-functional in that organism. Integration of the segment comprising the gene of interest may be selected if an appropriate marker is included within that segment. One or more nucleic acid sequences encoding appropriate signal peptides that are not naturally associated with a polypeptide to be expressed in a host cell of the invention can be incorporated into (expression) vectors. For example, a DNA sequence for a signal peptide leader can be fused in-frame to a nucleic acid of the invention so that the santalene synthase of the invention is initially translated as a fusion protein comprising the signal peptide. Depending on the nature of the signal peptide, the expressed polypeptide will be targeted differently. A secretory signal peptide that is functional in the intended host cells, for instance, enhances extracellular secretion of the expressed polypeptide. Other signal peptides direct the expressed polypeptide to certain organelles, like the chloroplasts, mitochondria and peroxisomes. The signal peptide can be cleaved from the polypeptide upon transportation to the intended organelle or from the cell. It is possible to provide a fusion of an additional peptide sequence at the amino or carboxyl terminal end of the polypeptide.

The term “gene construct” as used herein refers to polynucleotides comprising the polynucleotide of the invention and additional functional nucleic acid sequences. A gene construct according to the present invention is, preferably, a linear DNA molecule. Typically, a gene construct in accordance with the present invention may be a targeting construct which allows for random or site-directed integration of the targeting construct into genomic DNA. Such target constructs, preferably, comprise DNA of sufficient length for either homologous or heterologous recombination as described in detail below. In both cases, the construct must be, preferably, impeccable, with structures to control gene expression, such as a promoter, a site of transcription initiation, a site of polyadenylation, and a site of transcription termination.

Preferably, the method of the present invention comprises the step of obtaining said manufactured composition comprising at least one santalene.

The term “obtaining” as used herein refers to providing the composition comprising at least one santalene at any degree of purity. Accordingly, the composition may essentially consist of the at least one santalene in essentially pure form or may be a mixture comprising additional components besides the at least one santalene. Thus, the method of the invention may encompass one or more purification steps. The purification techniques which need to be applied depend on how the method of the present invention has been carried out. For example, if the method has been carried out in vitro, i.e. in reaction vials using isolated components such as isolated enzymes, adducts and auxiliary components such as reaction buffers, it will be understood that less purification is required. However, if the method is carried out in vivo, i.e. in a host cell as defined elsewhere herein, further purification and pre-treatment steps may be necessary. Typically, the host cells need to be harvested and the harvested cells will be lysed in order to release the composition comprising the at least one santalene from said cells. Subsequent purification steps shall remove the cell debris as well as aiming at purifying the at least one santalene from the remaining components. Moreover, if the steps are carried out in vivo in animals or plants, even further pre-treatment and/or purification steps may be required. The skilled person is well aware of suitable pre-treatment and/or purification steps depending on the given circumstances under which the method may be carried out. Purification techniques to be envisaged may be extraction techniques, chromatography, such as LC, GC or HPLC, size-exclusion chromatography, affinity chromatography, distillation, centrifugation, filtration and the like. Pre-treatment steps to be envisaged may be harvesting, heat treatment, ultra-sonic treatment, treatment with chemicals and/or enzymes, and the like. Particular preferred measures are described in the accompanying Examples, below.

Advantageously, the studies underlying the present invention revealed that a putative rice beta-santalene synthase was capable of synthesizing upon expression in bacteria, a mixture of santalenes comprising alpha-santalene and beta-santalene were beta santalene was synthesized in excess. So far, santalene synthases have been reported which pivotally produced alpha-santalene from farnesyl pyrophosphate with only minor amounts of beta-santalene. Moreover, the purification or enrichment of beta-santalene from compositions comprising essentially alpha-santalene is cumbersome. Thanks to the present invention and the surprising properties of the polypeptide exhibiting santalene synthase activity found in accordance with the present invention, santalene and, preferably, beta-santalene comprising compositions can be manufactured more efficiently, in particular, in recombinant manufacturing approaches.

The definitions and explanations of the terms made herein before apply mutatis mutandis to the following embodiments of the present invention except if specified otherwise.

In a preferred embodiment of the method of the present invention, the step of converting farnesyl pyrophosphate into at least one santalene is carried out in a host cell.

The term “host cell” as used herein relates to a prokaryotic or eukaryotic cell which is capable of converting farnesyl pyrophosphate into at least one santalene, wherein said conversion is carried out by a polypeptide exhibiting santalene synthase activity. Thus, the host cell of the invention is capable of expressing the polypeptide exhibiting santalene synthase activity. Preferably, said polypeptide exhibiting santalene synthase activity may be encoded by the heterologous polynucleotide or vector or gene construct of the invention. The host cell is, typically transformed with said heterologous polynucleotide, vector or gene construct such that the polypeptide exhibiting santalene synthase activity specified above can be expressed. The transformed vector or gene construct may be maintained as a non-integrated vector, for example, a plasmid, or alternatively, may be integrated into the host cell genome as specified elsewhere herein in more detail.

In one aspect of the invention, the host cell of the invention is a transgenic cell, i.e. transgenic for the nucleic acid encoding the santalene synthases of the invention, preferably a transgenic non-plant cell such as a transgenic microorganism cell.

A host cell according to the invention may be produced based on standard genetic and molecular biology techniques that are generally known in the art, e.g., as described in Sambrook, J., and Russell, D. W. “Molecular Cloning: A Laboratory Manual” 3d ed., Cold Spring Harbor Laboratory Press, Cold Spring Harbor, NY, (2001); and F. M. Ausubel et al, eds., “Current protocols in molecular biology”, John Wiley and Sons, Inc., New York (1987), and later supplements thereto.

Preferably, said host cell is selected from the group consisting of: a bacterial cell, a yeast cell, a fungal cell, an algal cell or a cyanobacterial cell, a non-human animal cell or a non-human mammalian cell, and a plant cell. More preferably, the host cell can be selected from any one of the following organisms:

### Bacteria:

The bacterial host cell can, for example, be selected from the group consisting of the genera Escherichia, Klebsiella, Helicobacter, Bacillus, Lactobacillus, Streptococcus, Amycolatopsis, Rhodobacter, Pseudomonas, Paracoccus, Lactococcus, Ensifer or Pantoea.

gram positive: Bacillus, Streptomyces. Useful gram positive bacterial host cells include, but are not limited to, a Bacillus cell, e.g., Bacillus alkalophius, Bacillus amyloliquefaciens, Bacillus brevis, Bacillus circulans, Bacillus clausii, Bacillus coagulans, Bacillus firmus, Bacillus Jautus, Bacillus lentus, Bacillus licheniformis, Bacillus megaterium, Bacillus pumilus, Bacillus stearothermophilus, Bacillus subtilis, and Bacillus thuringiensis. Most preferred, the prokaryote is a Bacillus cell, preferably, a Bacillus cell of Bacillus subtilis, Bacillus pumilus, Bacillus licheniformis, or Bacillus lentus.

Some other preferred bacteria include strains of the order Actinomycetales, preferably, Streptomyces, preferably Streptomyces spheroides (ATTC 23965), Streptomyces thermoviolaceus (IFO 12382), Streptomyces lividans or Streptomyces murinus or Streptoverticillum verticillium ssp. verticillium. Other preferred bacteria include Rhodobacter sphaeroides, Rhodomonas palustri, Streptococcus lactis. Further preferred bacteria include strains belonging to Myxococcus, e.g., M. virescens.

gram negative: Escherichia, Pseudomonas, Rhodobacter, Paracoccus, Ensifer or Pantoea species. Preferred gram negative bacteria are Escherichia coli, Pseudomonas sp., preferably, Pseudomonas purrocinia (ATCC 15958) or Pseudomonas fluorescens (NRRL B-11) or Pseudomonas denitrificans, Rhodobacter capsulatus or Rhodobacter sphaeroides, Paracoccus carotinifaciens, Paracoccus zeaxanthinifaciens, Pantoea ananatis, or Sinorhizobium meliloti also known as Ensifer meliloti.

### Fungi:

Aspergillus, Fusarium, Trichoderma. The host cell may be a fungal cell. “Fungi” as used herein includes the phyla Ascomycota, Basidiomycota, Chytridiomycota, and Zygomycota as well as the Oomycota and Deuteromycotina and all mitosporic fungi. Representative groups of Ascomycota include, e.g., Neurospora, Eupenicillium (=Penicillium), Emericella (=Aspergillus), Eurotium (=Aspergillus), and the true yeasts listed below. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g. Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

Some preferred fungi include strains belonging to the subdivision Deuteromycotina, class Hyphomycetes, e.g., Fusarium, Humicola, Tricoderma, Myrothecium, Verticillum, Arthromyces, Caldariomyces, Ulocladium, Embellisia, Cladosporium or Dreschlera, in particular Fusarium oxysporum (DSM 2672), Humicola insolens, Trichoderma resii, Myrothecium verrucana (IFO 6113), Verticillum alboatrum, Verticillum dahlie, Arthromyces ramosus (FERM P-7754), Caldariomyces fumago, Ulocladium chartarum, Embellisia alli or Dreschlera halodes. Other preferred fungi include strains belonging to the subdivision Basidiomycotina, class Basidiomycetes, e.g. Coprinus, Phanerochaete, Coriolus or Trametes, in particular Coprinus cinereus f. microsporus (IFO 8371), Coprinus macrorhizus, Phanerochaete chrysosporium (e.g. NA-12) or Trametes (previously called Polyporus), e.g. T. versicolor (e.g. PR4 28-A). Further preferred fungi include strains belonging to the subdivision Zygomycotina, class Mycoraceae, e.g. Rhizopus or Mucor, in particular Mucor hiemalis.

Yeast, Pichia, Saccharomyces: The fungal host cell may be a yeast cell. Yeast as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycesceae. The latter is comprised of four subfamilies, Schizosaccharomycoideae (e.g., genus Schizosaccharomyces), Nadsonioideae, Lipomycoideae, and Saccharomycoideae (e.g. genera Kluyveromyces, Pichia, and Saccharomyces). The basidiosporogenous yeasts include the genera Leucosporidim, Rhodosporidium, Sporidiobolus, Filobasidium, and Filobasidiella. Yeasts belonging to the Fungi Imperfecti are divided into two families, Sporobolomycetaceae (e.g., genera Sporobolomyces and Bullera) and Cryptococcaceae (e.g. genus Candida).

### Eukaryotes:

Eukaryotic host cells further include, without limitation, a non-human animal cell, a non-human mammal cell, an avian cell, reptilian cell, insect cell or a plant cell.

Most preferably, the host cell is a bacterial host cell, in particular, a Rhodobacter host cell.

In another preferred embodiment of the method of the present invention, the step of converting farnesyl pyrophosphate into at least one santalene is carried out in a non-human transgenic organism as defined herein below, or a non-vertebrate transgenic organism such as a plant or a microorganism.

The term “transgenic non-human organism” as used herein refers to an organism which has been genetically modified in order to comprise the polynucleotide, vector or gene construct of the present invention. Said genetic modification may be the result of any kind of homologous or heterologous recombination event, mutagenesis or gene editing process. Accordingly, the transgenic non-human organism shall differ from its non-transgenic counterpart in that it comprises the non-naturally occurring (i.e. heterologous) polynucleotide, vector or gene construct in its genome. Non-human organisms envisaged as transgenic non-human organisms in accordance with the present invention are, preferably, multi-cellular organisms. Moreover, the non-human organisms are, preferably, animals or plants. Preferred animals are mammals, in particular laboratory animals such as rodents, e.g., mice, rats, rabbits or the like, or farming animals such as sheep, goat, cows, horses or the like. Preferred plants are crop plants or vegetables, in particular, selected from the group consisting of Arabidopsis spp., Nicotiana spp, Cichorum intybus, Lactuca sativa, Mentha spp, Artemisia annua, tuber forming plants, oil crops, e.g. Brassica spp. or Brassica napus, flowering plants (angiosperms) which produce fruits, and trees.

Methods for the production of transgenic non-human organisms are well known in the art; see, e.g. Lee-Yoon Low et al., Transgenic Plants: Gene constructs, vector and transformation method. 2018. DOI.10.5772/intechopen.79369; Pinkert, C. A. (ed.) 1994. Transgenic animal technology: A laboratory handbook. Academic Press, Inc., San Diedo, Calif.; Monastersky G. M. and Robl, J. M. (ed.) (1995) Strategies in Transgenic Animal Science. ASM Press. Washington D.C); Sambrook, loc.cit, Ausubel, loc.cit).

Preferably, said non-human transgenic organism is a plant or a non-human animal to be sacrificed. Accordingly, it accordance with the latter, methods of treating animals are not encompassed within the methods of the present invention.

The present invention also relates to a composition comprising a mixture of beta-santalene and alpha-santalene with an excess of beta-santalene, obtainable by the method of the present invention. Preferably, said beta-santalene is present in the composition in a relative amount to total santalenes of at least about 50%, at least about 60%, at least about 70%, preferably between about 50% and about 80%, between about 60% and about 75%, between about 65% and about 70%, more preferably at least about 67%. Preferably, the composition is substantially free of beta-farnesene. The composition of the invention is preferably a lipophilic composition.

In another aspect of the invention, said inventive composition comprises, in addition Sesquithujene, preferably in an amount of at least 0.01%.

Surprisingly, it was found that the presence of sesquithujene in the santalene compositions of the invention resulted in an improved olfactory property of the inventive santalene composition. Compared to typical santalene compositions, the inventive compositions have a stronger woody note. So far, sesquithujene had not been reported to have any effect on olfactory properties.

In yet another aspect of the invention, the ratio of bergamotene, preferably trans-alpha bergamotene, to beta-santalene in the compositions produced with the help of the santalene synthase of the invention is 1:2, 1:3, 1:4, 1:5, or 1:10, or less.

In another aspect of the invention, the composition produced is substantially free of any one of cis-alpha-Bergamotene ((AS Nr. 18252-46-5), (E)-beta-Farnesene (CAS Nr. 18794-84-8), trans-beta-Bergamotene (CAS Nr.15438-94-5) or beta-Bisabolene (CAS Nr.495-61-4), or all of these, preferably substantially free of (E)-beta-Farnesene (CAS Nr. 18794-84-8).

In one embodiment, the composition of the invention substantially consists of beta-Santalene, alpha-Santalene, trans-alpha-Bergamotene, Sesquithujene and epi-beta-Santalene, preferably beta-Santalene, alpha-Santalene, trans-alpha-Bergamotene and Sesquithujene.

The present invention relates to the use of the aforementioned polypeptide, heterologous polynucleotide, vector or gene construct, host cell or non-human transgenic organism for the manufacture of a composition comprising at least one santalene, preferably, beta-santalene, more preferably a mixture of beta-santalene and alpha-santalene, even more preferably a mixture of beta-santalene and alpha-santalene and sesquithujene. The beta-santalene, alpha santalene, sesquithujene or bergamotene produced by the inventive methods, or the compositions of the invention may be used in flavour or fragrance applications, in cosmetic uses, in pharmaceuticals, as insect repellent or insect attractant, or in agriculture e.g. for crop protection or animal raising.

Yet, the present invention relates to a method for manufacturing a composition comprising at least one santalol, preferably beta-santalol, comprising:
a) producing a composition comprising at least one santalene by the aforementioned method of the present invention; and
b) oxidising said at least one santalene, preferably, beta-santalene, into the respective alcohol in order to manufacture a composition comprising at least one santalol, preferably, beta-santalol.

Preferably, the composition produced in step a) of the inventive methods for manufacturing a composition comprising at least one santalol, preferably beta-santalol, is substantially free or at least largely reduced in the amounts of (E)-beta-Farnesene.

Preferably, said method is further comprising the step of obtaining said composition comprising at least one santalol. More preferably, said composition comprises a mixture of alpha-santalol and beta-santalol with an excess of beta-santalol.

The term “santalol” as used herein refers to an alcoholic sesquiterpene. The term encompasses alpha santalol (Z)-5-(2,3-Dimethyltricyclol [2.2.1.02,6] hept-3-yl)-2-methylpent-2-en-1-ol; CAS number: 115-71-9; molecular formula C15H24O) and beta santalol (2Z)-2-Methyl-5-[(1S,2R,4R)-2-methyl-3-methylidene bicyclo [2.2.1] heptan-2-yl] pent-2-en-1-ol; CAS number: 77-42-9; molecular formula C₁₅H₂₄O).

In the aforementioned method of the present invention, the composition comprising at least one santalene is generated in a first step, step (a), as described by the method of the invention elsewhere herein. In a subsequent step, step (b), said at least one santalene comprised in the composition is oxidized in order to manufacture a composition comprising at least one santalol as referred to above. In one aspect of the invention the oxidation of the at least one santalene is, preferably, carried out enzymatically. Suitable enzymes that are capable oxidizing santalene and, thus, converting it into santalol are well known in the art. Preferably, cytochrome P450 monooxygenases (CYPs) are used for oxidizing santalene into santalol. More preferably, the CYP is selected from the group consisting of: CYP76F37v1, CYP76F37v2, CYP76F38v1, CYP76F38v2, CYP76F39v1, CYP76F41, CYP76F42, CYP76F39v2, CYP76F40, and CYP736A167. Yet, the oxidation may also be carried out chemically.

Step (b) of the aforementioned method may also be carried out in vivo or ex vivo, typically, dependent on how step (a) is carried out. Thus, if step (a) is carried out in vivo, e.g., in a host cell or in a non-human transgenic organism as specified elsewhere herein, it is preferably envisaged that step (b) is carried out in vivo as well and, preferably, in the same host cell or non-human transgenic organism. Typically, the host cell or non-human transgenic organism shall be capable of carrying out the oxidation of the santalene into santalol. Preferably, the said host cell or non-human transgenic organism may, thus, express a CYP as specified above. To this end, a heterologous polynucleotide encoding said CYP or a vector or gene construct comprising such a polynucleotide may be present in the host cell or non-human transgenic organism. How such heterologous polynucleotides, vectors or gene constructs may be introduced into the said host cell or non-human transgenic organism is well known in the art and described elsewhere herein in detail.

Alternatively, step b) of the method for manufacturing a composition comprising at least one santalol is carried out by a chemical process rather than an enzymatic one. Preferably, the chemical oxidation to santalol(s) is performed as disclosed in WO 2021/063831.

The invention also relates to compositions comprising beta-santalol and alpha-santalol produced from a precursor composition comprising both beta-santalene and alpha santalene produced by the methods of the invention, wherein the beta-santalol herein is present in greater amounts on a w/w basis than the alpha santalol due to a surplus beta santalene content in the precursor composition. The beta-santalol to alpha santalol ratio in these compositions is greater than 1, preferably the ratio is at least 1.1, 1.2, 1.3, 1.4, 1.5, 1.6, 1.7, 1.8, 1.9 or at least 2. The ratio of beta-santalol to alpha-santalol may be at least 3:1, preferably at least 4:1, more preferably at least 5:1, even more preferably 6:1, yet even more preferably at least 7:1, most preferably at least 8:1 and even at least 9:1. In one aspect of the invention, the ratio is not greater than 100:1.

The invention, therefore, also contemplates a method for producing a composition with a surplus of beta-santalol over alpha-santalol without the need to a) diminish the alpha-santalene content before the conversion to alpha-santalol and/or b) to increase the beta-santalol content after the conversion from santalenes by distillation or other means, wherein the method comprises the steps of producing a composition with a surplus of beta-santalene over alpha-santalene by the methods of the invention, and in one or more subsequent steps oxidising the beta-santalene to beta-santalol and the alpha-santalene to alpha-santalol.

This conversion of the santalenes may be done biosynthetically and/or chemically to their respective alcohols. Following the conversion to santalols, purification steps like a distillation to remove other compounds may be included, and if desired the ratio of beta-santalol to alpha-santalol may be altered by distillation, but a composition with more beta-santalol than alpha-santalol can be achieved without further alterations of the beta-santalol to alpha-santalol ratio following the provision of the composition with beta-santalene in excess of alpha-santalene by the use of the improved beta santalene synthases. One aspect of the invention hence is a method for the production of a composition comprising beta-santalol in excess to alpha-santalol, wherein the method comprises the steps of producing a composition with a surplus of beta-santalene over alpha-santalene by the methods of the invention, and in one or more subsequent steps oxidising the beta-santalene to beta-santalol and the alpha-santalene to alpha-santalol and wherein a distillation of santalols following the oxidation of santalenes is performed for purification of the santalols without increasing the beta-santalol content over the alpha-santalol content substantially.

Also the invention relates to compositions comprising beta-santalol in excess to alpha-santalol produced by any of the methods of the invention, with the improved beta santalene synthases of the invention or with the host cells of the invention, optionally with the sum of bergamotols in the compositions being less than 10% (w/w) or even less than 8% (w/w). In another aspect, the inventive compositions comprising beta-santalol in excess to alpha-santalol produced by any of the methods of the invention, with the improved beta santalene synthases of the invention or with the host cells of the invention comprise less than 3% epi-beta-santalol.

Using the inventive compositions comprising santalenes and sesquithujene in the production of santalol has additional benefits. As the santalene composition comprising sesquithujene has an improved olfactory property, the resulting santalol composition is also be improved in its olfactory properties.

One aspect of the invention relates to a polypeptide with santalene synthase activity of the invention producing beta-santalene in excess of alpha-santalene, a nucleic acid encoding such, an expression cassette comprising such nucleic acids, host cells comprising such expression cassettes, methods of the invention and compositions produced with the inventive enzymes and methods, comprising beta-santalene and alpha-santalene and/or beta-santalol and alpha santalol with a ratio of beta-santalene to alpha-santalene or the ratio of beta-santalol to alpha-santalol, respectively, of at least equal to or greater than 1.3, 1.5 or 2.

The invention also pertains to a method for the manufacture of sesquithujene, or a composition comprising sesquithujene, comprising:
a) producing a composition comprising at least one santalene and sesquithujene by the method of the invention; and, optionally,
b) isolating the sesquithujene from the composition.

The present invention also pertains to a kit for the manufacture of a composition comprising at least one santalene comprising the aforementioned polypeptide, heterologous polynucleotide, vector or gene construct, host cell or non-human transgenic organism, wherein the composition preferably is substantially free of beta-farnesene and/or comprises sesquithujene.

The term “kit” as used herein refers to a collection of components required for carrying out the method of the present invention for the manufacture of a composition at least one santalene.

The kit shall include any of the aforementioned components either as a single component or any combinations thereof. Typically, the components of the kit are provided in separate containers or within a single container. The container also typically comprises instructions for carrying out the method of the present invention for manufacture of a composition at least one santalene. Moreover, the kit may, preferably, comprise further components which are necessary for carrying out the method of the invention such as incubation reagents, cultivation media, washing solutions, solvents, and/or reagents or means required for purification of the composition at least one santalene.

Also, the present invention contemplates a non-human host cell expressing a polypeptide exhibiting santalene synthase activity from the aforementioned heterologous polynucleotide, vector or gene construct. Preferably, said host cell is selected from the group consisting of: a bacterial cell, a yeast cell, a fungal cell, an algal cell or a cyanobacterial cell, a non-human animal cell or a non-human mammalian cell, and a plant cell. More preferably, said non-human host cell produces at least one santalene, preferably, beta-santalene.

The present invention relates to a non-human transgenic organism expressing a polypeptide exhibiting santalene synthase activity from a heterologous polynucleotide, the vector or gene construct as referred to before. Preferably, said non-human transgenic organism is a plant or a non-human animal. More preferably, said non-human host cell produces at least one santalene, preferably, beta-santalene.

The non-human host cell, non-human transgenic organism and methods of the invention may comprise the polypeptide exhibiting santalene synthase activity of the invention and in addition one or more further polypeptide exhibiting santalene synthase activity, including those known in the art under the names CiCaSSy (QNV69588) or Santalum album santalene synthase (E3W202) and/or those santalene synthases included in the international patent application with the filing number PCT/EP2021/064642.

Finally, the invention pertains to an aroma composition comprising at least one composition according to claim 10, or a composition as defined in claim 11, or a composition produced by any of the methods of claims 1 to 8, and any of (ii) to (iv):

(ii) at least one aroma chemical (X) other than a compound selected from the group consisting of beta-Santalene, alpha-Santalene, trans-alpha-Bergamotene, Sesquithujene and epi-beta-Santalene, or
(iii) at least one non-aroma chemical carrier, or
(iv) both of (ii) and (iii).

In a preferred embodiment, the at least one composition according to claim 10 or the composition as defined in claim 11 or produced by any of the methods of claims 1 to 8, is present in the range of ≥0.01 wt. % to ≤70.0 wt. %, based on the total weight of the aroma composition.

In another preferred embodiment, the at least one non-aroma chemical carrier (iii) is selected from surfactants, oil components, antioxidants, deodorant-active agents, or solvents.

In a still further preferred embodiment, the composition is selected from perfume compositions, body care compositions, hygiene articles, cleaning compositions, textile detergent compositions, compositions for scent dispensers, foods, food supplements, pharmaceutical compositions, or crop protection compositions.

The following embodiments are particular preferred embodiments envisaged in accordance with the present invention. All definitions an explanations of the terms made above apply mutatis mutandis.

Embodiment 1. A method for the manufacture of a composition comprising at least one santalene comprising the step of converting farnesyl pyrophosphate into at least one santalene, wherein said conversion is carried out by a polypeptide exhibiting santalene synthase activity, wherein said polypeptide comprises

(ii) an amino acid sequence selected from the group consisting of:
a) an amino acid sequence as shown in any of SEQ ID NO: 1, 18, or 21 to 28;
b) an amino acid sequence which is at least 60%, at least 70%, at least 80%, at least 85%, at least 90%, at least 95%, at least 98% or at least 99% identical to the amino acid sequences as shown in any of SEQ ID NO: 1, 18, or 21 to 28;
c) an amino acid sequence encoded by a nucleic acid sequence as shown in any one of SEQ ID NOs: 2 or 3 or 19;
d) an amino acid sequence encoded by a nucleic acid sequence which is at least 60%, at least 70%, at least 80%, at least 85%, at least 90%, at least 95%, at least 98% or at least 99% identical to the nucleic acid sequence as shown in any one of SEQ ID NOs: 2 or 3 or 19; and
e) an amino acid sequence of a fragment of any one of (a) to (d), said fragment encoding a polypeptide exhibiting a santalene synthase activity; or
   (ii) an amino acid sequence selected from the group consisting of:
a) an amino acid sequence as shown in any one of SEQ ID NO: 4 to 17;
b) an amino acid sequence which is at least 60%, at least 70%, at least 80%, at least 85%, at least 90%, at least 95%, at least 98% or at least 99% identical to the amino acid sequences as shown in SEQ ID NO: 4 to 17; and
c) an amino acid sequence of a fragment of (a) or (b), said fragment encoding a polypeptide exhibiting a santalene synthase activity.

Embodiment 2: The method of embodiment 1, wherein said at least one santalene is beta-santalene.

Embodiment 3: The method of embodiment 1 or 2, wherein said at least one santalene is a mixture of beta-santalene and alpha-santalene with an excess of beta-santalene and, preferably, the composition also comprises sesquithujene.

Embodiment 4.: The method of embodiment 3, wherein said beta-santalene is present in the composition in a relative amount to total santalenes of at least about 50%, at least about 60%, at least about 70%, preferably, between about 50% and about 80%, between about 60% and about 75%, between about 65% and about 70%, more preferably at least about 67%.

Embodiment 5: The method of any one of embodiments 1 to 4, wherein the step of converting farnesyl pyrophosphate into at least one santalene is carried out in a host cell.

Embodiment 6: The method of embodiment 5, wherein said host cell is selected from the group consisting of: a bacterial cell, a yeast cell, a fungal cell, an algal cell or a cyanobacterial cell, a non-human animal cell or a non-human mammalian cell, and a plant cell.

Embodiment 7: The method of any one of embodiments 1 to 4, wherein the step of converting farnesyl pyrophosphate into at least one santalene is carried out in a non-human transgenic organism.

Embodiment 8: The method of embodiment 7, wherein said non-human transgenic organism is a plant or a non-human animal to be sacrificed.

Embodiment 9: The method of any one of embodiments 5 to 8, wherein said polypeptide exhibiting santalene synthase activity is encoded by a heterologous polynucleotide, a vector or a gene construct.

Embodiment 10: The method of any one of embodiments 1 to 9, further comprising the step of obtaining the composition comprising at least one santalene.

Embodiment 11: A composition comprising a mixture of beta-santalene and alpha-santalene with an excess of beta-santalene, and preferably also comprising sesquithujene obtainable by the method of any one of embodiments 1 to 10.

Embodiment 12: The composition of embodiment 11, wherein said beta-santalene is present in the composition in a relative amount to total santalenes of at least about 50%, at least about 60%, at least about 70%, preferably, between about 50% and about 80%, between about 60% and about 75%, between about 65% and about 70%, more preferably at least about 67%.

Embodiment 13: Use of the polypeptide as defined in any one of embodiments 1 to 4, the heterologous polynucleotide, the vector or gene construct as defined in claim in embodiment 9, the host cell as defined in embodiment 5 or 6 or the non-human transgenic organism as defined in embodiment 7 or 8 for the manufacture of a composition comprising at least one santalene, preferably, beta-santalene, more preferably, a mixture of beta-santalene and alpha-santalene, even more preferably a mixture of beta-santalene and alpha-santalene and sesquithujene, most preferably, as defined in embodiments 11 or 12.

Embodiment 14: The use of embodiment 13, wherein said santalene is used for producing flavours, fragrances, pharmaceuticals or cosmetics.

Embodiment 15: A method for manufacturing a composition comprising at least one santalol, preferably beta-santalol, comprising:
a) producing a composition comprising at least one santalene by the method of any one of embodiments 1 to 10; and
b) oxidising said at least one santalene, preferably, beta-santalene, into the respective alcohol in order to manufacture a composition comprising at least one santalol, preferably, beta-santalol.

Embodiment 16: The method of embodiment 15, further comprising the step of obtaining said composition comprising at least one santalol.

Embodiment 17: The method of embodiment 15 or 16, wherein said composition comprises a mixture of alpha-santalol and beta-santalol with an excess of beta-santalol.

Embodiment 18: A kit for the manufacture of a composition comprising at least one santalene comprising the polypeptide as defined in embodiment 1, the heterologous polynucleotide, the vector or gene construct as defined in claim in embodiment 9, the host cell as defined in embodiment 5 or 6 or the non-human transgenic organism as defined in embodiment 7 or 8, wherein the composition is substantially free of beta-farnesene, and preferably comprises sesquithujene.

Embodiment 19: A non-human host cell expressing a polypeptide exhibiting santalene synthase activity from the heterologous polynucleotide, the vector or gene construct as defined in claim in embodiment 9.

Embodiment 20: The non-human host cell of embodiment 19, wherein said host cell is selected from the group consisting of: a bacterial cell, a yeast cell, a fungal cell, an algal cell or a cyanobacterial cell, a non-human animal cell or a non-human mammalian cell, and a plant cell.

Embodiment 21: The non-human host cell of embodiment 19 or 20, wherein said non-human host cell produces at least one santalene, preferably, beta-santalene.

Embodiment 22: A non-human transgenic organism expressing a polypeptide exhibiting santalene synthase activity from the heterologous polynucleotide, the vector or gene construct as defined in claim in embodiment 9.

Embodiment 23: The non-human transgenic organism of embodiment 22, wherein said non-human transgenic organism is a plant or a non-human animal.

Embodiment 24: The non-human transgenic organism of embodiment 22 or 23, wherein said non-human host cell produces at least one santalene, preferably, beta-santalene.

All references cited throughout this specification are herewith incorporated by reference in their entireties or with respect to the specifically mentioned disclosure content.

### FIGURES

FIG. 1: Schematic drawing of the vector p-m-SPppa-MBP-OmBSS-mpmii alt.

FIG. 2: Production of beta-santalene in E. coli. GC-MS analysis of dodecane phases of BL21-DE3-pMEV-pAC-OmBSS (A) and BL21-DE3-pMEV-pACYC-DUET-1 (B).

FIG. 3: Production of beta-santalene in E. coli. GC analysis of dodecane phase ofRs265-9c/p-m-SPppa-OmBSS-mpmii alt. Percentages of total santalenes: alpha-santalene: 25.7%; beta-santalene: 67.3%; trans-alpha bergamotene 7.0%.

FIG. 4: Amino acid alignment of Oryza santalene synthase having SEQ ID NO: 1 and proteins from other Oryza species. The alignment was made by using MUSCLE (Multiple Sequence Comparison by Log-Expectation) and the standard parameters.

The following sequences are referred to throughout the specification and in the accompanying sequence protocol:

SEQ ID NO: 1: putative Oryza meridionalis beta-santalene synthase (OmBSS) protein

SEQ ID NO: 2: synthetic coding sequence OmBSS for E. coli

SEQ ID NO: 3: synthetic coding sequence OmBSS for Rhodobacter

SEQ ID NO: 4: NM49164 Oryza sativa amino acid sequence

SEQ ID NO: 5: A0AOD9Z7P2_90RYZ Uncharacterized protein Oryza glumipatula

SEQ ID NO: 6: A2XGY8_ORYSI Uncharacterized protein Oryza sativa subsp. indica

SEQ ID NO: 7: A0AOEONVD9_ORYRU Uncharacterized protein Oryza rufipogon

SEQ ID NO: 8: A0AON7KHA7|A0AON7KHA7_ORYSJ Os03g0361700 protein (Fragment) Oryza sativa subsp. japonica

SEQ ID NO: 9: Q10L24_ORYSJ Terpene synthase family, metal binding domain containing protein, expressed Oryza sativa subsp. japonica SEQ ID NO: 10: 11PBH4_ORYGL Uncharacterized protein Oryza glaberrima

SEQ ID NO: 11: C7J0Q6_ORYSJ Os03g0361700 protein Oryza sativa subsp. japonica

SEQ ID NO: 12: A0AOEOGMM5_ORYNI Uncharacterized protein Oryza nivara

SEQ ID NO: 13: A0AOD3FIR1_90RYZ Uncharacterized protein Oryza barthii

SEQ ID NO: 14: A3AI64_ORYSJ Uncharacterized protein Oryza sativa subsp. japonica

SEQ ID NO: 15: AOAOEONVE4_ORYRU Uncharacterized protein Oryza rufipogon

SEQ ID NO: 16: A0A0E0DOX2_90RYZ Uncharacterized protein Oryza meridionalis

SEQ ID NO: 17: A0AOD3FIR4_90RYZ Uncharacterized protein Oryza barthii

SEQ ID NO: 18: synthetic fusion protein of a maltose binding protein MBP and Oryza meridionalis beta-santalene synthase (OmBSS)

SEQ ID NO: 19: synthetic coding sequence encoding a synthetic fusion protein of a maltose binding protein and Oryza meridionalis beta-santalene synthase (OmBSS)

SEQ ID NO: 20: GRXCX4W motif

SEQ ID NO: 21: synthetic santalene synthase SynBSS1

SEQ ID NO: 22: synthetic santalene synthase SynBSS2

SEQ ID NO: 23: synthetic santalene synthase SynBSS3

SEQ ID NO: 24: synthetic santalene synthase SynBSS4

SEQ ID NO: 25: synthetic santalene synthase SynBSS5

SEQ ID NO: 26: synthetic santalene synthase SynBSS6

SEQ ID NO: 27: synthetic santalene synthase SynBSS7

SEQ ID NO: 28: synthetic santalene synthase SynBSS8

### EXAMPLES

The Examples shall merely illustrate the invention. They shall not, whatsoever, be construed as limiting the scope.

### Example 1: Identifying a Rice Santalene Synthase

A protein sequence, which is shown in UniProt accession number AOAOEODOX4 (SEQ ID NO: 1) was extracted from the Oryza meridionalis genome (https://www.genome.jp/dbget-bin/www_bget?uniprot: A0A0E0D0X4_9ORYZ).

A0A0E0D0X4:
MSGSKVISVLDTKVLAVKGGTMTQPVAAATTGRACSPSLWGDFFVTYIPP
KPQRSEEWMRERVDWLKMQVGCKILKTINVPYTVMLVDVLERLHIDNHFR
DEIATALQHVFHHDEQQKAAAGFDDGDQLHLESLRFRLLRQHGFWVSADV
FDKFKDSTGCFRESLSTDARGLLSLYNAAHLAMPGEAALDDAIAFSRRSL
QSLQGALRSPMAEQVSRALDIPLPRAPKLLETMHYITEYEQEAAHDGMVL
ELARLDFELVRSLYLKELKALSLWWRQLYGSVQLSYARDCLVESYFWTCA
MFHGEDYSRARIIFAKVFQLMTMTDDIYDIHATLEECYKFNKAIQRWDKS
AVSILPEYLRNFYIRILNDFDEMEDSLEPDEKHRMSYVKSSFKQQSEYYL
REAQWSSDKHMPSFAEHLDVSSMSIGYPTMAVVVLLCARDGDGAAASMEA
SEWAPSLVRAGGEVTRFLNDIASYKTGKSGKDAASTIECYMAERGVGGEE
AVAAVAALVESAWRTINRACVEMDPNLLPAARLLVNLATTPEVIYFGGRD
GYTVGADLKGLVTALFLDPLRV

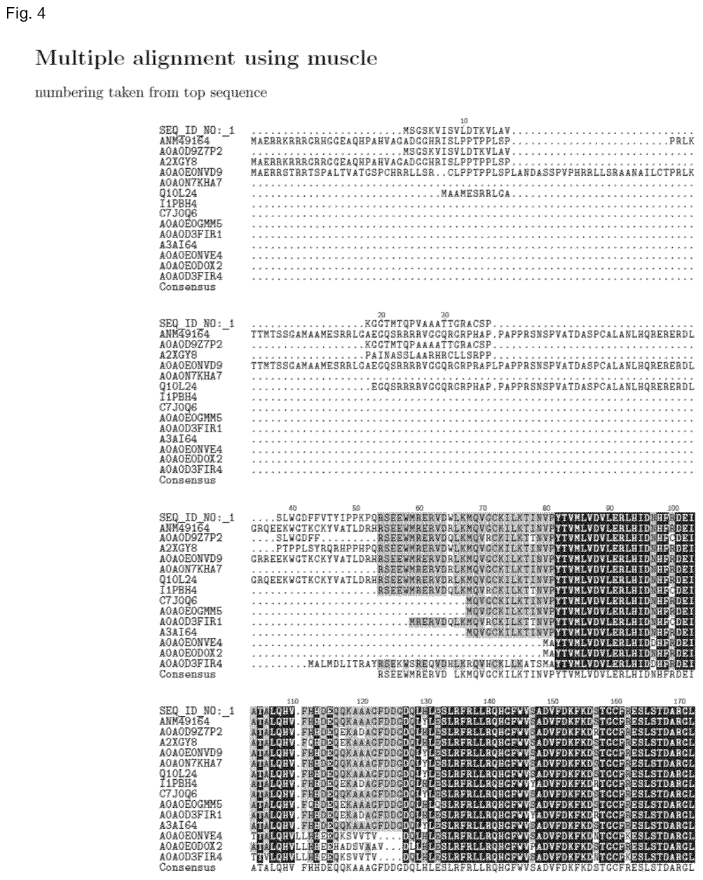

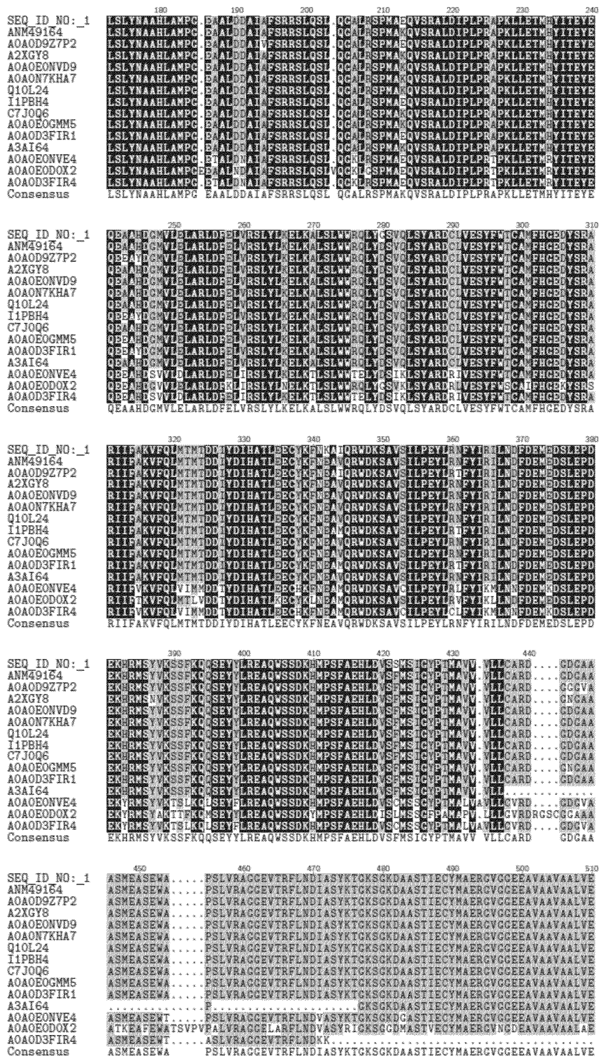

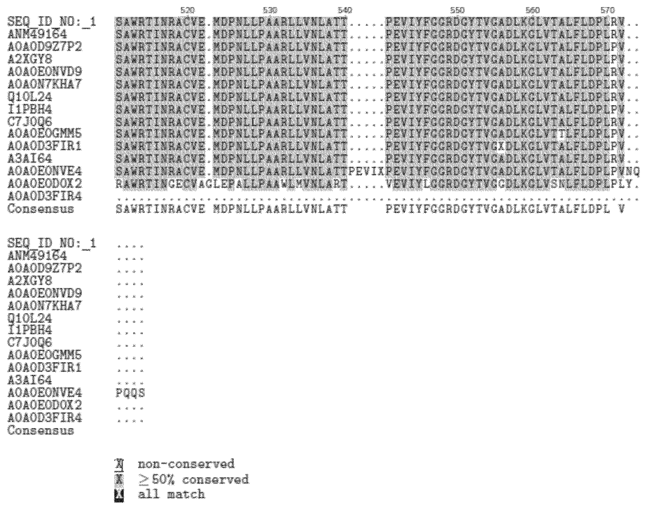

In a BLASTP analysis (nr database), the best blast hit (98.4% identical) is with a sequence which is annotated as a (+)-germacrene D synthase from Oryza sativa japonica. +/−7 other sequences map within 90% identity, these sequences are found in other rice variants (Oryza sativa indica etc.). A MUSCLE alignment of SEQ ID NO: 1 and related sequences is shown in FIG. 4. The best hits were 95% with an uncharacterized Oryza glumipatula protein.

However, sequence identity to known santalene synthase proteins is very limited: CiCaSSy (QNV69588): 30.9% Santalum album santalene synthase (E3W202): 27.4%.

To test the use of this protein, it was expressed in Escherichia coli and in Rhodobacter sphaeroides.

### Example 2: Cloning for Expression in E. coli.

The following sequence was synthesized using a standard sequence service provider, cloned in expression vector pACYC-Duet-1 (Novagen) using BamHI and NotI restriction sites. The plasmid was labelled pAC-OsBSS.

Synthetic sequence encoding A0A0E0D0X4 for E coli expression
(SEQ ID NO: 2):
ggatccgATGAGCGGTTCCAAAGTTATTTCTGTTCTGGATACCAAAGTTCTGGCGGTTAAAGG
CGGCACCATGACCCAGCCGGTTGCTGCTGCTACCACCGGTCGTGCTTGTAGCCCATCTCT
GTGGGGTGACTTCTTCGTGACCTACATTCCGCCGAAACCGCAGCGTTCTGAAGAATGGAT
GCGTGAACGTGTTGACTGGCTGAAAATGCAGGTAGGCTGTAAAATCCTGAAAACCATCAAC
GTGCCGTACACCGTTATGCTGGTAGATGTTCTGGAACGTCTGCACATCGATAACCACTTCC
GTGATGAAATCGCGACTGCACTGCAGCACGTGTTCCACCACGATGAACAGCAGAAAGCAG
CGGCTGGTTTCGATGACGGTGACCAGCTGCACCTGGAGAGCCTGCGCTTCCGTCTGCTGC
GTCAGCACGGTTTCTGGGTATCTGCGGACGTGTTTGACAAATTCAAAGACTCTACCGGCTG
CTTCCGTGAATCCCTGTCTACCGACGCTCGCGGCCTGCTGTCTCTGTACAACGCCGCTCA
CCTGGCTATGCCGGGTGAAGCGGCTCTGGATGACGCTATTGCGTTCTCCCGTCGTTCTCT
GCAGTCTCTGCAGGGTGCGCTGCGTTCCCCAATGGCGGAACAGGTTTCCCGCGCACTGG
ACATCCCGCTGCCGCGTGCTCCGAAACTGCTGGAAACCATGCACTACATCACCGAATACG
AACAGGAAGCCGCGCACGATGGCATGGTTCTGGAACTGGCACGTCTGGATTTCGAACTGG
TGCGTTCTCTCTACCTGAAAGAACTGAAAGCTCTGTCTCTGTGGTGGCGTCAGCTGTATGG
CTCTGTTCAGCTGTCCTACGCTCGTGATTGCCTGGTTGAAAGCTACTTCTGGACCTGTGCA
ATGTTCCACGGTGAAGATTACTCCCGTGCACGTATCATCTTCGCTAAAGTTTTCCAGCTGAT
GACTATGACTGATGACATCTACGACATCCACGCGACCCTGGAAGAATGCTACAAATTTAAC
AAAGCAATCCAGCGTTGGGATAAATCTGCGGTGTCTATTCTGCCGGAATACCTGCGTAACT
TCTACATCCGTATCCTGAATGACTTTGACGAAATGGAAGATAGCCTGGAACCGGACGAAAA
ACATCGCATGTCTTACGTTAAATCTAGCTTCAAACAGCAGAGCGAATACTACCTGCGTGAA
GCGCAGTGGTCCTCTGACAAACACATGCCGTCCTTCGCTGAACACCTGGACGTTAGCAGC
ATGTCTATCGGTTACCCGACTATGGCAGTTGTTGTGCTGCTGTGTGCACGTGATGGCGAC
GGCGCGGCTGCTTCTATGGAAGCGTCTGAATGGGCGCCGTCCCTTGTTCGTGCGGGTGG
TGAAGTTACTCGCTTTCTGAACGACATCGCATCTTACAAAACCGGCAAATCTGGTAAAGAT
GCTGCAAGCACCATCGAATGCTATATGGCTGAACGTGGCGTGGGCGGTGAAGAAGCGGT
GGCGGCTGTTGCGGCGCTGGTCGAATCCGCATGGCGCACCATCAACCGCGCATGCGTTG
AAATGGACCCGAACCTGCTGCCGGCGGCCCGTCTGCTGGTTAACCTGGCCACCACTCCG
GAAGTGATCTATTTCGGTGGTCGTGACGGTTACACCGTTGGTGCGGACCTGAAAGGTCTG
GTGACCGCTCTGTTCCTGGACCCGCTGCGTGTTtaagcggccgc

The plasmid was introduced in E. coli BL21 DE3 harbouring pMEV, which has been described in Schmidt et al. 2017 (Scientific Reports|7:862 |DOI: 10.1038/s41598-017-00893-3).

Transformants were selected on LB-agar plates+chloramphenicol (30 ug/ml)+kanamycin (30 ug/ml)+1% glucose. A positive transformant (tested by miniprep and restriction digestion) was labelled E. coli BL21-DE3-pMEV-pAC-OmBSS. Using the same method, a control strain harbouring pMEV and pACYC-DUET-1 was created, and labelled BL21-DE3-pMEV-PACYC-DUET-1.

### Example 3: Production of Beta-Santalene in E. coli

The strain BL21-DE3-pMEV-pAC-OmBSS and BL21-DE3-pMEV-PACYC-DUET-1 were inoculated in 5 ml LB liquid medium+chloramphenicol (30 ug/ml)+kanamycin (30 ug/ml)+1% glucose and incubated overnight at 37° C. and 250 rpm. Next day, the cultures were diluted 1:25 in 10 ml 2xYT medium +chloramphenicol (30 ug/ml) +kanamycin (30 ug/ml) and grown at 37° C. and 250 rpm until A600 was 0.5. Subsequently, 1 mM IPTG was added as inducer, 1 ml of n-dodecane was added to capture the products and the cultures were further incubated for 24 hours at 28° C. 250 rpm.

For GC-MS analysis the dodecane was separated from the cultures by centrifugation and diluted 200 times with ethyl acetate. 2 μL were analysed by GC/MS using a gas chromatograph as described in detail by Cankar et al. (2015).

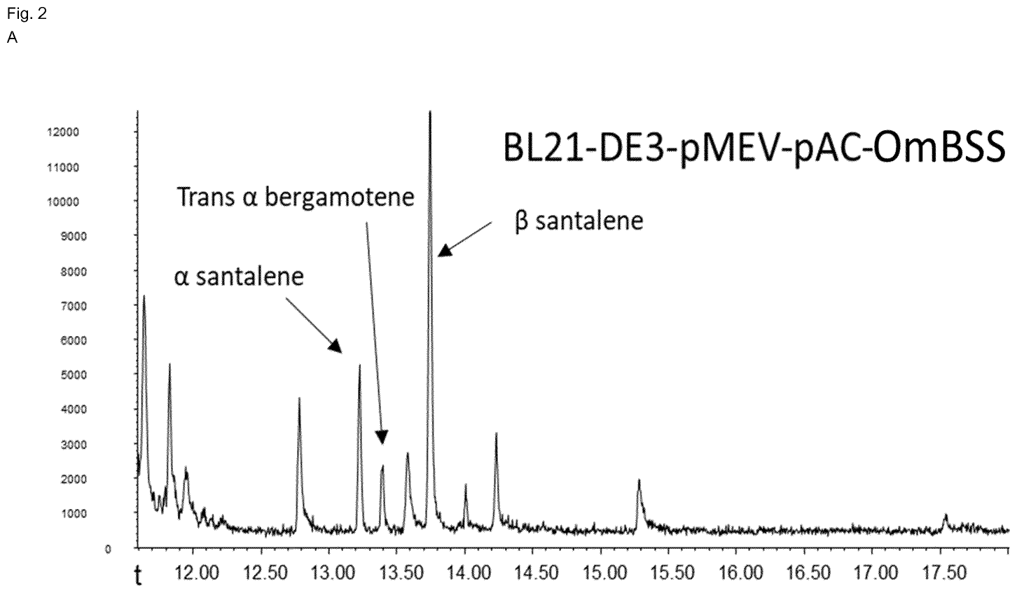

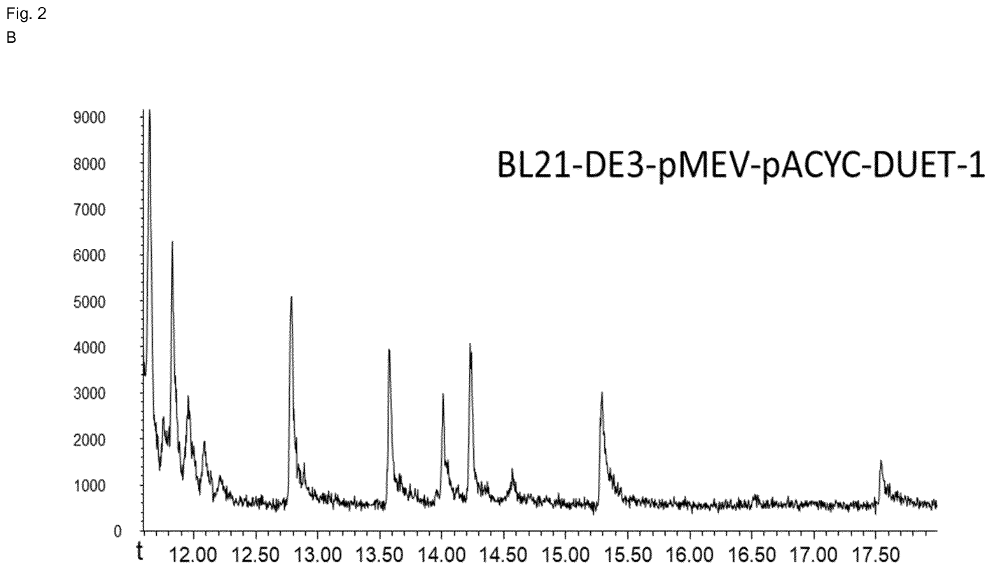

Surprisingly, when compared to BL21-DE3-pMEV-PACYC-DUET-1, the BL21-DE3-pMEV-pAC-OmBSS produced alpha-santalene, trans-alpha bergamotene and beta-santalene. The major santalene product of OmBSS in this system was found to be β-santalene (FIG. 2).

### Example 4: Construct for Expressing OmBSS in Rhodobacter Sphaeroides.

The following synthetic DNA was ordered from Genscript.

Synthetic gene OmBSS for Rhodobacter expression (SEQ ID NO: 3):
AAGCTTATCATGTCGGGCTCGAAGGTGATCTCGGTCCTCGACACCAAGGTCCTGGCTGTC
AAGGGCGGCACGATGACGCAGCCGGTGGCCGCAGCGACCACGGGGCGCGCCTGCTCGC
CCAGCCTCTGGGGCGATTTCTTCGTGACCTATATCCCGCCGAAGCCGCAGCGGTCGGAAG
AATGGATGCGCGAGCGGGTGGACTGGCTGAAGATGCAGGTGGGCTGCAAGATCCTGAAG
ACAATCAACGTGCCCTATACCGTGATGCTGGTGGACGTGCTGGAGCGTCTGCATATCGAC
AACCATTTCCGCGACGAGATCGCCACCGCCCTGCAGCATGTGTTCCATCACGACGAGCAG
CAGAAGGCCGCAGCTGGCTTCGATGACGGCGACCAGCTGCACCTTGAGAGCCTGCGCTT
TCGGCTCCTGCGGCAGCACGGCTTCTGGGTGTCGGCCGACGTCTTCGACAAGTTCAAGGA
CAGCACCGGCTGCTTCCGCGAGTCGCTGTCGACCGATGCCCGGGGGCTGCTCAGTCTCT
ACAACGCCGCGCACCTCGCCATGCCGGGCGAGGCCGCCCTTGACGATGCGATCGCCTTC
TCGCGGCGCTCCCTTCAGTCGCTGCAGGGCGCGCTGCGCAGCCCGATGGCCGAGCAGGT
GTCGCGCGCCTTGGATATTCCGCTGCCGCGCGCGCCCAAGCTCCTGGAGACGATGCACT
ACATCACCGAGTACGAGCAGGAGGCGGCCCATGACGGCATGGTGCTCGAACTCGCGCGC
CTCGACTTCGAGCTCGTTCGGTCGCTCTATCTCAAGGAGCTGAAGGCGCTCTCGCTCTGG
TGGCGGCAATTGTACGGCTCCGTGCAGCTCAGCTATGCCCGCGACTGCCTCGTTGAGAGC
TACTTCTGGACCTGCGCGATGTTCCACGGGGAGGACTATTCGCGCGCGCGGATCATCTTC
GCCAAGGTCTTCCAGCTGATGACCATGACCGACGATATCTACGACATCCACGCGACGCTC
GAGGAATGCTACAAGTTCAACAAGGCCATCCAGCGCTGGGACAAGTCGGCCGTGTCGATC
CTCCCCGAATACTTACGCAACTTCTACATCCGCATCCTCAATGACTTCGATGAGATGGAGG
ATAGCCTTGAGCCCGACGAGAAGCATCGGATGTCCTATGTCAAATCCTCGTTCAAGCAGCA
GAGCGAATATTACCTGAGGGAGGCGCAGTGGAGCTCCGACAAGCACATGCCCTCGTTCGC
GGAGCATCTGGATGTGTCTAGCATGAGCATCGGCTATCCCACGATGGCGGTGGTGGTCCT
GCTCTGCGCCCGCGATGGAGACGGGGCGGCCGCCTCGATGGAAGCGAGCGAGTGGGCG
CCGTCGCTGGTGCGGGCGGGCGGTGAGGTGACACGCTTCCTGAACGACATCGCGAGCTA
CAAGACCGGCAAATCCGGGAAGGACGCGGCGTCCACGATCGAGTGTTACATGGCCGAGC
GCGGCGTGGGCGGCGAGGAGGCCGTCGCCGCGGTGGCGGCGCTCGTGGAGTCGGCCT
GGCGCACCATCAATCGGGCCTGCGTCGAGATGGACCCGAACCTCCTGCCCGCGGCCCGA
CTGCTGGTGAACCTGGCGACGACGCCTGAGGTCATCTATTTCGGCGGCCGCGACGGGTA
TACGGTCGGCGCCGATCTGAAAGGGCTCGTCACCGCACTGTTTCTCGACCCGCTCCGGGT
GTGAggatcc

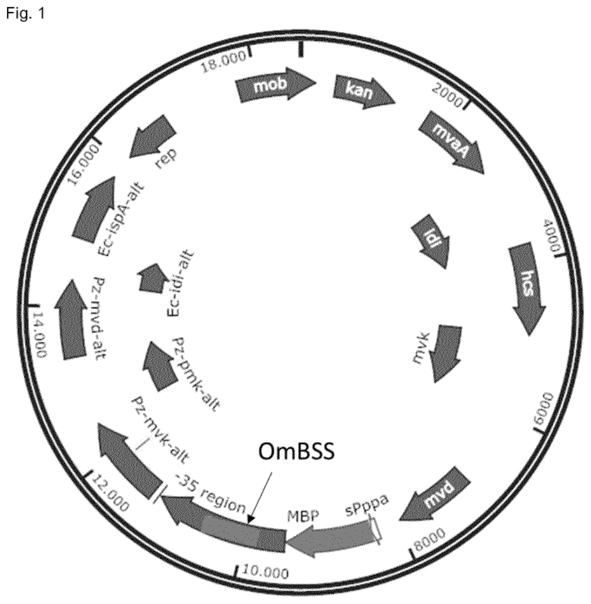

This fragment was cloned in plasmid p-m-SPppa-MBP-CiCaSSy-mpmii-alt from patent application U.S. Pat. No. 20,200,010822A1, using HindHI and BamHI restriction sites. The ligation mixture was transformed into E. coli S17-1 cells. Transfer of p-m-SPppa-MBP-OmBSS-mpmii alt (FIG. 1) from S17-1 to R. sphaeroides Rs265-9c by conjugation was performed using standard procedures (Patent U.S. Pat. No. 9,260,709 B2). The fusion protein expressed is shown in SEQ ID NO: 18.

### Example 5: Production of Beta-Santalene in Rhodobacter Sphaeroides

Seed cultures of Rs265-9c/p-m-SPppa-MBP-OmBSS-mpmii alt were performed in 100 ml shake flasks without baffles with 20 ml RS102 medium with 100 mg/L neomycin and a loop of glycerol stock. Seed culture flasks were grown for 72 hours at 30° C. in a shaking incubator with an orbit of 50 mm at 110 rpm.

Shake flask production experiments were performed in 300 ml shake flasks with 2 bottom baffles. Twenty ml of RS102 medium (as described in U.S. Pat. No. 20,200,010822A1) and neomycin to a final concentration of 100 mg/L were added to the flask together with 2 ml of sterile n-dodecane. The volume of the inoculum was adjusted to obtain a final OD600 value of 0.05 in 20 ml medium. The flasks were kept for 72 hours at 30° C. in a shaking incubator with an orbit of 50 mm at 110 rpm. Shake flask experiments were performed in duplicates.

For GC-MS analysis the dodecane was separated from the culture by centrifugation and diluted 10 times with acetone.

Gas chromatography was performed on a Shimadzu GC2010 Plus equipped with a Restek RTX-5Sil MS capillary column (30 m×0.25 mm, 0.5 pm). The injector and FID detector temperatures were set to 280° C. and 300° C., respectively. Gas flow through the column was set at 40 ml/min. The oven initial temperature was 160° C., increased to 180° C. at a rate of 2° C./min, further increased to 300° C. at a rate of 50° C./min, and held at that temperature for 3 min. Injected sample volume was 1 μL with a 1:50 split-ratio, and the nitrogen makeup flow was 30 ml/min.

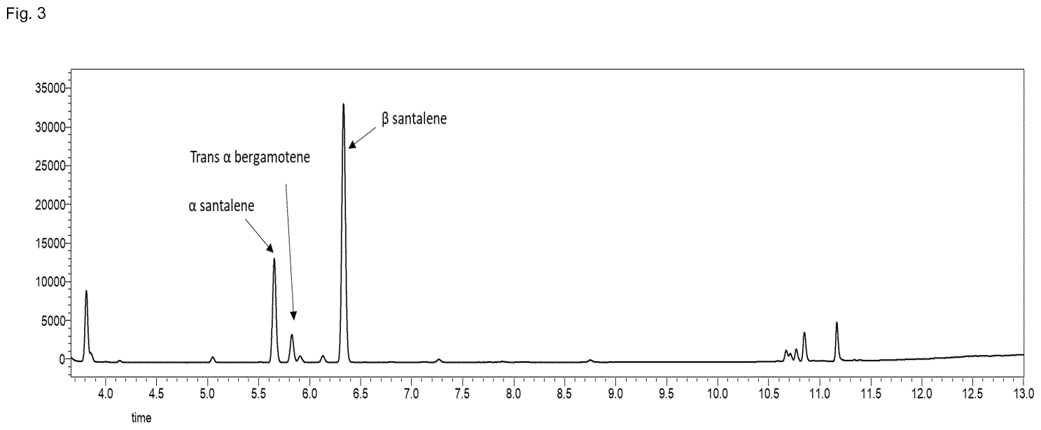

Compounds found in the dodecane layer from the Rs265-9c/p-m-SPppa-MBP-OmBSS-mpmii alt culture were identified according to their retention time and quantified by integrating their peak area in the chromatogram (FIG. 3). Analysis of peak areas revealed that 26% of total santalenes was alpha-santalene, 7% was trans-alpha-bergamotene and 67% was beta-santalene.

A second experiment was conducted to analyse the produced terpene substance in further detail, and in particular for such terpenes that are known from other santalene synthases. The results of the GC analysis of the compounds found in the dodecane layer are shown in Table I. Surprisingly, it was found that some terpenes known to be produced by known santalene synthases were not produced, and that the santalene synthase of the invention had a novel and very different product profile, including the production of sesquithujene.

TABLE I
List of expected and found compounds in the dodecane
layer after culturing the OmBSS expressing cells:

| | | | Area | Area % |
|---|---|---|---|---|
| Compound | CAS Nr. | RT | OmBSS | OmBSS |
| Sesquithujene | 58319-06-5 | 16.16 | 3686 | 1 |
| cis-alpha-Bergamotene | 18252-46-5 | 16.81 | ND | ND |
| alpha-Santalene | 512-61-8 | 16.94 | 129719 | 24 |
| trans-alpha- | 13474-59-4 | 17.31 | 32802 | 6 |
| Bergamotene | | | | |
| epi-beta-Santalene | 25532-78-9 | 17.63 | 7973 | 1 |
| (E)-beta-Farnesene | 18794-84-8 | 17.74 | ND | ND |
| beta-Santalene | 511-59-1 | 17.94 | 336068 | 62 |
| trans-beta- | 15438-94-5 | 18.54 | ND | ND |
| Bergamotene | | | | |
| beta-Bisabolene | 495-61-4 | 19.07 | ND | ND |

Substances in italics were expected to be present but not produced by OmBSS. It was confirmed that other known santalene synthases would produce detectable amounts of these in the same set-up.

RT=retention time Found RI=experimental RI, Lit.RI=RI from publications, ND not detected.

### Example 6: Olfactory Comparison of Novel Santalene Compositions of the Invention With Known Santalene Compositions

The santalene composition comprising the santalenes and sesquithujene was produced as described in the previous examples. For comparison, santalene compositions of the state of the art were produced as described in WO 2018/160066.

When the inventive santalene composition was smelled in comparison to the known santalene compositions, the composition of the invention had an improved olfactory property with an increased woody note.

The compositions comprising the santalenes and sesquithujene can be used in aroma compositions like known compositions comprising santalenes are used, and the inventive compositions comprising santalol can be used in aroma compositions like known Santalol compositions are used.

Suitable aroma compositions are, for example, but not limited to perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions and crop protection compositions.

Perfume compositions can be selected from fine fragrances, air fresheners in liquid form, gel-like form or a form applied to a solid carrier, aerosol sprays, scented cleaners, perfume candles and oils, such as lamp oils or oils for massage.

Examples for fine fragrances are perfume extracts, Eau de Parfums, Eau de Toilettes, Eau de Colognes, Eau de Solide and Extrait Parfum.

The inventive compositions can also be used for flavouring.

### Advantageous Compositions

The inventive compositions comprising a mix of santalenes and sesquithujene were formulated in the perfume compositions according to Tables II and Ill and were labelled as compound A.

The inventive mixtures of santalenes and sesquithujene were formulated as compositions according to Tables II and III. Mixtures indicated in Table I were labelled as “compound A”, in Table II and III.

TABLE II
Compositions 1A and 1B

| | 1A | 1B |
|---|---|---|
| Lactone C10 gamma (5-hexyloxolan-2-one) | 2 | 2 |
| Bourgeonal (3-(4-tert-butylphenyl)propanal) | 2 | 2 |
| Citronellol | 3 | 3 |
| Aldehyde C-14 (5-heptyloxolan-2-one) | 3 | 3 |
| Allyl heptylate | 4 | 4 |
| Amber core (1-(2-tert-butylcyclohexyl)oxybutan-2-ol) | 4 | 4 |
| Ethyl-2-methyl butyrate | 4 | 4 |
| Geranyl acetate | 5 | 5 |
| Helional (3-(1,3-benzodioxol-5-yl)-2-methylpropanal) | 10 | 10 |
| Manzanate (ethyl 2-methylpentanoate) | 10 | 10 |
| Amberwood (ethoxymethoxycyclododecane) | 10 | 10 |
| Hexyl acetate | 11 | 11 |
| Benzyl salicylate | 12 | 12 |
| Magnolan (2,4-dimethyl-4,4a,5,9b- | 15 | 15 |
| tetrahydroindeno[1,2-d][1,3]dioxine) | | |
| Verdox (2-tert-butylcyclohexyl) acetate) | 25 | 25 |
| Bergamot oil bergaptene free | 25 | 25 |
| Linalool | 30 | 30 |
| Dipropylene glycol | 45 | 45 |
| Iso E Super (Tetramethyl acetyloctahydronaphthalenes) | 110 | 110 |
| Pyranol (4-methyl-2-(2-methylpropyl)oxan-4-ol) | 170 | 170 |
| Hedione (methyl 3-oxo-2-pentylcyclopentaneacetate) | 200 | 200 |
| Galaxolide 50% IPM (1,3,4,6,7,8-hexahydro- | 300 | 300 |
| 4,6,6,7,8,8-hexamethylcyclopenta(g)-2-benzopyran | | |
| 50% in isopropyl myristate) | | |
| compound A | 10 | 20 |
| Total (parts by weight) | 1020 | 1050 |

TABLE III
Compositions 2A and 2B

| | 2A | 2B |
|---|---|---|
| Raspberry ketone (4-(4-hydroxyphenyl)butan-2-one) | 4 | 4 |
| Vanitrope (2-ethoxy-5-prop-1-enylphenol) | 6 | 6 |
| Cyclamen aldehyde (at least 90% | 10 | 10 |
| 2-methyl-3-(p-isopropylphenyl)- | | |
| propionaldehyde; secondary component: | | |
| 5% 3-(p-cumenyl)-2-methylpropionic acid) | | |
| Bicyclononalactone (3,4,4a,5,6,7,8,8a- | 10 | 10 |
| octahydrochromen-2-one) | | |
| Aldehyde C-14 (5-heptyloxolan-2-one) | 14 | 14 |
| Ethylvanillin (3-ethoxy-4-hydroxybenzaldehyde) | 16 | 16 |
| Heliotropine (1,3-benzodioxole-5-carbaldehyde) | 20 | 20 |
| Iso E Super (tetramethyl acetyloctahydronaphthalenes) | 20 | 20 |
| Sandela (3-[5,5,6-trimethylbicyclo[2.2.1]hept- | 30 | 30 |
| 2-yl]cyclohexan-1-ol) | | |
| Vanillin isobutyrate ((4-formyl-2-methoxyphenyl) 2- | 40 | 40 |
| methylpropanoate) | | |
| Aldehyde C-18 (5-pentyloxolan-2-one) | 50 | 50 |
| Benzyl salicylate | 60 | 60 |
| Hexyl cinnamic aldehyde (2-(phenylmethylidene)octanal) | 70 | 70 |
| Hedione (methyl 3-oxo-2-pentylcyclopentaneacetate) | 130 | 130 |
| Pyranol (4-methyl-2-(2-methylpropyl)oxan-4-ol) | 150 | 150 |
| Ethylene brassylate (1,4-dioxacycloheptadecane- | 170 | 170 |
| 5,17-dione) | | |
| Galaxolide 50% IPM (1,3,4,6,7,8-hexahydro- | 200 | 200 |
| 4,6,6,7,8,8-hexamethylcyclopenta(g)-2-benzopyran | | |
| 50% in isopropyl myristate) | | |
| compound A | 10 | 20 |
| Total (parts by weight) | 1020 | 1050 |

Composition according to Table II and Table III namely 1A, 1B, 2A, 2B could be included in various compositions selected from the group consisting of Deo pump spray, Clean hair-conditioner, Face wash gel, Foam bath concentrate, Hair gel, Self-foaming bodywash, Sprayable sun care emulsion, Sprayable sun protection emulsion, Emollient facial gel, 2-phases oil foam bath, Shampoos, Shower bath, Hydro-alcoholic AP/Deo pump spray, Aerosol, Aqueous/alcoholic AP/Deo roll-on, Styling Gel Type “Out of Bed”, Shaving Foam, Sensitive skin Baby shampoo, Body wash for Sensitive Skin, Gloss Enhancing Shampoo for Sensitive Scalp, Deo Stick, Baby Wipe, After shave balm, Face Gel, Face Day Care Cream, Face Cleanser, Body lotion, Sun Care SPF50+, Sprayable Lotion, Hand dish cleaner—regular, Hand dish cleaner—concentrate, Sanitary cleaner—concentrate, All-purpose cleaner, Anti-bacterial fabric softener, Detergent composition, Powder detergent composition and Liquid detergent composition.

A person skilled in the art may be well versed with the various general formulations for the above-mentioned products.

Compositions 1A, 1B, 2A and 2B can, for example, be formulated in specific formulations as disclosed in IP.com Number: IPCOM000258614D entitled New Aroma Chemicals pages 6 to 46, Table 1 to Table D13, wherein the “Fragrance Composition 1A” is replaced by identical amounts of compositions 1A, 1B, 2A or 2B.

1-16. (canceled)

17. A method for the manufacture of a composition comprising at least one santalene comprising the step of converting farnesyl pyrophosphate into at least one santalene, wherein said conversion is carried out by at least one polypeptide exhibiting santalene synthase activity, wherein said at least one polypeptide comprises an amino acid sequence selected from the group consisting of:
a) an amino acid sequence as shown in any of SEQ ID NO: 1, 18, 21, 22, 23, 24, 25, 26, 27, or 28;
b) an amino acid sequence which is at least 60%, at least 70%, at least 80%, at least 85%, at least 90%, at least 95%, at least 98% or at least 99% identical to the amino acid sequences as shown in any of SEQ ID NO: 1, 18, or 21, 22, 23, 24, 25, 26, 27, or 28;
c) an amino acid sequence encoded by a nucleic acid sequence as shown in any one of SEQ ID NOs: 2 or 3 or 19;
d) an amino acid sequence encoded by a nucleic acid sequence which is at least 60%, at least 70%, at least 80%, at least 85%, at least 90%, at least 95%, at least 98% or at least 99% identical to a polypeptide encoded by the nucleic acid sequence as shown in any one of SEQ ID NOs: 2 or 3 or 19; and
e) an amino acid sequence of a fragment of any one of (a) to (d), said fragment encoding a polypeptide exhibiting santalene synthase activity.

18. The method of claim 17, wherein said at least one santalene is beta-santalene.

19. The method of claim 17, wherein said at least one santalene is a mixture of beta-santalene and alpha-santalene with an excess of beta-santalene.

20. The method of claim 19, wherein said beta-santalene is present in the composition in a relative amount to total santalenes of at least about 50%, at least about 60%, or at least about 70%.

21. The method of claim 20, wherein said beta-santalene is present in the composition in a relative amount to total santalenes of between about 50% and about 80%, between about 60% and about 75%, or between about 65% and about 70%.

22. The method of claim 17, wherein the step of converting farnesyl pyrophosphate into said at least one santalene is carried out in a host cell.

23. The method of claim 17, wherein the step of converting farnesyl pyrophosphate into said at least one santalene is carried out in a non-vertebrate transgenic organism.

24. The method of claim 17, wherein the composition further comprises sesquithujene in detectable amounts and is substantially free of any one of cis-alpha-Bergamotene ((AS Nr. 18252-46-5), (E)-beta-Farnesene (CAS Nr. 18794-84-8), trans-beta-Bergamotene (CAS Nr.15438-94-5) or beta-Bisabolene (CAS Nr.495-61-4), or all of these.

25. The method of claim 17, wherein said polypeptide exhibiting santalene synthase activity is encoded by a heterologous polynucleotide, a vector, or a gene construct.

26. A composition comprising a mixture of beta-santalene and alpha-santalene with an excess of beta-santalene obtainable by the method of claim 17, wherein the composition further comprises sesquithujene in detectable amounts and is substantially free of any one of cis-alpha-Bergamotene ((AS Nr. 18252-46-5), (E)-beta-Farnesene (CAS Nr. 18794-84-8), trans-beta-Bergamotene (CAS Nr.15438-94-5) or beta-Bisabolene (CAS Nr.495-61-4), or all of these.

27. A composition comprising a mixture of beta-santalene and alpha-santalene with an excess of beta-santalene obtainable by the method of claim 17, wherein said beta-santalene is present in the composition in a relative amount to total santalenes of at least about 50%, at least about 60%, or at least about 70%, wherein the composition is substantially free of beta-farnesene and/or comprises sesquithujene.

28. The composition of claim 27, wherein said beta-santalene is present in the composition in a relative amount to total santalenes of between about 50% and about 80%, between about 60% and about 75%, or between about 65% and about 70%.

29. A method for manufacturing a composition comprising at least one santalol, preferably beta-santalol, comprising:
a) producing a composition comprising at least one santalene by the method of claim 17; and
b) oxidising said at least one santalene, preferably, beta-santalene, into the respective alcohol in order to manufacture a composition comprising at least one santalol.

30. A kit for the manufacture of a composition comprising at least one santalene comprising the polypeptide as defined in claim 17.

31. A non-human host cell expressing a polypeptide exhibiting santalene synthase activity from the heterologous polynucleotide, the vector or gene construct as defined in claim 24.

32. A non-human or non-vertebrate transgenic organism expressing a polypeptide exhibiting santalene synthase activity from the heterologous polynucleotide, the vector or gene construct as defined in claim 25.

33. An aroma composition comprising,
i) the composition comprising at least one santalene produced by methods of claim 17; and any of (ii) to (iv):
ii) at least one aroma chemical (X) other than a compound selected from the group consisting of beta-Santalene, alpha-Santalene, trans-alpha-Bergamotene, Sesquithujene and epi-beta-Santalene, or
iii) at least one non-aroma chemical carrier, or
iv) both of (ii) and (iii).

34. An aroma composition comprising,
i) The composition of claim 26; and any of (ii) to (iv):
ii) at least one aroma chemical (X) other than a compound selected from the group consisting of beta-Santalene, alpha-Santalene, trans-alpha-Bergamotene, Sesquithujene and epi-beta-Santalene, or
iii) at least one non-aroma chemical carrier, or
iv) both of (ii) and (iii).


SEQUENCE LISTING (28 sequences)

SEQ ID NO: 1 - protein, 572 aa, Oryza meridionalis
MSGSKVISVLDTKVLAVKGGTMTQPVAAATTGRACSPSLWGDFFVTYIPPKPQRSEEWMRERVDWLKMQVGCKILKTINVPYTVMLVDVLERLHIDNHFRDEIATALQHVFHHDEQQKAAAGFDDGDQLHLESLRFRLLRQHGFWVSADVFDKFKDSTGCFRESLSTDARGLLSLYNAAHLAMPGEAALDDAIAFSRRSLQSLQGALRSPMAEQVSRALDIPLPRAPKLLETMHYITEYEQEAAHDGMVLELARLDFELVRSLYLKELKALSLWWRQLYGSVQLSYARDCLVESYFWTCAMFHGEDYSRARIIFAKVFQLMTMTDDIYDIHATLEECYKFNKAIQRWDKSAVSILPEYLRNFYIRILNDFDEMEDSLEPDEKHRMSYVKSSFKQQSEYYLREAQWSSDKHMPSFAEHLDVSSMSIGYPTMAVVVLLCARDGDGAAASMEASEWAPSLVRAGGEVTRFLNDIASYKTGKSGKDAASTIECYMAERGVGGEEAVAAVAALVESAWRTINRACVEMDPNLLPAARLLVNLATTPEVIYFGGRDGYTVGADLKGLVTALFLDPLRV

SEQ ID NO: 2 - DNA, 1734 bp, synthetic construct
  misc_feature 1..1734: note = synthetic coding sequence
  source 1..1734: mol_type = other DNA
ggatccgatgagcggttccaaagttatttctgttctggataccaaagttctggcggttaaaggcggcaccatgacccagccggttgctgctgctaccaccggtcgtgcttgtagcccatctctgtggggtgacttcttcgtgacctacattccgccgaaaccgcagcgttctgaagaatggatgcgtgaacgtgttgactggctgaaaatgcaggtaggctgtaaaatcctgaaaaccatcaacgtgccgtacaccgttatgctggtagatgttctggaacgtctgcacatcgataaccacttccgtgatgaaatcgcgactgcactgcagcacgtgttccaccacgatgaacagcagaaagcagcggctggtttcgatgacggtgaccagctgcacctggagagcctgcgcttccgtctgctgcgtcagcacggtttctgggtatctgcggacgtgtttgacaaattcaaagactctaccggctgcttccgtgaatccctgtctaccgacgctcgcggcctgctgtctctgtacaacgccgctcacctggctatgccgggtgaagcggctctggatgacgctattgcgttctcccgtcgttctctgcagtctctgcagggtgcgctgcgttccccaatggcggaacaggtttcccgcgcactggacatcccgctgccgcgtgctccgaaactgctggaaaccatgcactacatcaccgaatacgaacaggaagccgcgcacgatggcatggttctggaactggcacgtctggatttcgaactggtgcgttctctctacctgaaagaactgaaagctctgtctctgtggtggcgtcagctgtatggctctgttcagctgtcctacgctcgtgattgcctggttgaaagctacttctggacctgtgcaatgttccacggtgaagattactcccgtgcacgtatcatcttcgctaaagttttccagctgatgactatgactgatgacatctacgacatccacgcgaccctggaagaatgctacaaatttaacaaagcaatccagcgttgggataaatctgcggtgtctattctgccggaatacctgcgtaacttctacatccgtatcctgaatgactttgacgaaatggaagatagcctggaaccggacgaaaaacatcgcatgtcttacgttaaatctagcttcaaacagcagagcgaatactacctgcgtgaagcgcagtggtcctctgacaaacacatgccgtccttcgctgaacacctggacgttagcagcatgtctatcggttacccgactatggcagttgttgtgctgctgtgtgcacgtgatggcgacggcgcggctgcttctatggaagcgtctgaatgggcgccgtcccttgttcgtgcgggtggtgaagttactcgctttctgaacgacatcgcatcttacaaaaccggcaaatctggtaaagatgctgcaagcaccatcgaatgctatatggctgaacgtggcgtgggcggtgaagaagcggtggcggctgttgcggcgctggtcgaatccgcatggcgcaccatcaaccgcgcatgcgttgaaatggacccgaacctgctgccggcggcccgtctgctggttaacctggccaccactccggaagtgatctatttcggtggtcgtgacggttacaccgttggtgcggacctgaaaggtctggtgaccgctctgttcctggacccgctgcgtgtttaagcggccgc

SEQ ID NO: 3 - DNA, 1734 bp, synthetic construct
  misc_feature 1..1734: note = synthetic coding sequence
  source 1..1734: mol_type = other DNA
aagcttatcatgtcgggctcgaaggtgatctcggtcctcgacaccaaggtcctggctgtcaagggcggcacgatgacgcagccggtggccgcagcgaccacggggcgcgcctgctcgcccagcctctggggcgatttcttcgtgacctatatcccgccgaagccgcagcggtcggaagaatggatgcgcgagcgggtggactggctgaagatgcaggtgggctgcaagatcctgaagacaatcaacgtgccctataccgtgatgctggtggacgtgctggagcgtctgcatatcgacaaccatttccgcgacgagatcgccaccgccctgcagcatgtgttccatcacgacgagcagcagaaggccgcagctggcttcgatgacggcgaccagctgcaccttgagagcctgcgctttcggctcctgcggcagcacggcttctgggtgtcggccgacgtcttcgacaagttcaaggacagcaccggctgcttccgcgagtcgctgtcgaccgatgcccgggggctgctcagtctctacaacgccgcgcacctcgccatgccgggcgaggccgcccttgacgatgcgatcgccttctcgcggcgctcccttcagtcgctgcagggcgcgctgcgcagcccgatggccgagcaggtgtcgcgcgccttggatattccgctgccgcgcgcgcccaagctcctggagacgatgcactacatcaccgagtacgagcaggaggcggcccatgacggcatggtgctcgaactcgcgcgcctcgacttcgagctcgttcggtcgctctatctcaaggagctgaaggcgctctcgctctggtggcggcaattgtacggctccgtgcagctcagctatgcccgcgactgcctcgttgagagctacttctggacctgcgcgatgttccacggggaggactattcgcgcgcgcggatcatcttcgccaaggtcttccagctgatgaccatgaccgacgatatctacgacatccacgcgacgctcgaggaatgctacaagttcaacaaggccatccagcgctgggacaagtcggccgtgtcgatcctccccgaatacttacgcaacttctacatccgcatcctcaatgacttcgatgagatggaggatagccttgagcccgacgagaagcatcggatgtcctatgtcaaatcctcgttcaagcagcagagcgaatattacctgagggaggcgcagtggagctccgacaagcacatgccctcgttcgcggagcatctggatgtgtctagcatgagcatcggctatcccacgatggcggtggtggtcctgctctgcgcccgcgatggagacggggcggccgcctcgatggaagcgagcgagtgggcgccgtcgctggtgcgggcgggcggtgaggtgacacgcttcctgaacgacatcgcgagctacaagaccggcaaatccgggaaggacgcggcgtccacgatcgagtgttacatggccgagcgcggcgtgggcggcgaggaggccgtcgccgcggtggcggcgctcgtggagtcggcctggcgcaccatcaatcgggcctgcgtcgagatggacccgaacctcctgcccgcggcccgactgctggtgaacctggcgacgacgcctgaggtcatctatttcggcggccgcgacgggtatacggtcggcgccgatctgaaagggctcgtcaccgcactgtttctcgacccgctccgggtgtgaggatcc

SEQ ID NO: 4 - protein, 653 aa, Oryza sativa
  REGION 1..653: note = putative protein
MAERRKRRRGRHGGEAQHPAHVAGADGGHRISLPPTPPLSPPRLKTTMTSSGAMAAMESRRLGAEGQSRRRRVGGQRGRPHAPPAPPRSNSPVATDASPCALANLHQRERERDLGRQEEKWGTKCKYVATLDRHRSEEWMRERVDRLKMQVGCKILKTINVPYTVMLVDVLERLHIDNHFRDEIATALQHVFHHDEQQKAAAGFDDGDQLYLESLRFRLLRQHGFWVSADVFDKFKDSTGCFRESLSTDARGLLSLYNAAHLAMPGEAALDDAIAFSRRSLQSLQGALRSPMAKQVSRALDIPLPRAPKLLETMHYITEYEQEAAHDGMVLELARLDFELVRSLYLKELKALSLWWRQLYDSVQLSYARDCLVESYFWTCAMFHGEDYSRARIIFAKVFQLMTMTDDIYDIHATLEECYKFNEAVQRWDKSAVSILPEYLRNFYIRILNDFDEMEDSLEPDEKHRMSYVKSSFKQQSEYYLREAQWSSDKHMPSFAEHLDVSFMSIGYPTMAVVVLLCARDGDGAAASMEASEWAPSLVRAGGEVTRFLNDIASYKTGKSGKDAASTIECYMAERGVGGEEAVAAVAALVESAWRTINRACVEMDPNLLPAARLLVNLATTPEVIYFGGRDGYTVGADLKGLVTALFLDPLRV

SEQ ID NO: 5 - protein, 563 aa, Oryza glumipatula
  REGION 1..563: note = putative protein
MSGSKVISVLDTKVLAVKGGTMTQPAAAATTGRACSPSLWGDFFRSEEWMRERVDQLKMQVRCKILKTTNVPYTVMLVDVLERLHIDNHFCDEIATALQHVFHHDEQEKADAGFDDGDQLHLESLRFRLLRQHGFWVSADVFDKFKDRTGCFRESLSTDARGLLSLYNAAHLAMPGEAALDDAIVFSRRSLQSLQGALRSPMAEQVSRALDIPLPRAPKLLETMHYITEYEQEEAYDGMVLELARLDFELVRSLYLKELKALSLWWRQLYDSVQLSYARDCLVESYFWTCAMFHGEDYSRARIIFAKVFQLMTMTDDIYDIHATLEECYKFNEAIQRWDKSAVSILPEYLRTFYIRILNDFDEMEDSLEPDEKHRMSYVKSSFKQQSEYYLREAQWSSDKHMPSFAEHLDVSFMSIGYPTMAVVVLLCARDGGGVAASMEASEWAPSLVRAGGEVTRFLNDIASYKTGKSGKDAASTIECYMAERGVGGEEAVAAVAALVESAWRTINRACVEMDPNLLPAARLLVNLATTPEVIYFGGRDGYTVGADLKGLVTALFLDPLPV

SEQ ID NO: 6 - protein, 596 aa, Oryza sativa
  REGION 1..596: note = putative protein
  source 1..596: note = Oryza sativa Indica Group
MAERRKRRRGRRGGEAQHPAHVAGADGGHRISLPPTPPLSPPAINASSLAARHRCLLSRPPPTPPLSYRQRHPPHPQRSEEWMRERVDWLKMQVGCKILKTINVPYTVMLVDVLERLHIDNHFRDEIATALQHVFQHDEQEKAAAGFDDGDQLHLESLRFRLLRQHGFWVSADVFDKFKDSTGCFRESLSTDARGLLSLYNAAHLAMPGEAALDDAIAFSRRSLQSLQGALRSPMAKQVSRALDIPLPRAPKLLETMHYITEYEQEAAHDGMVLELARLDFELVRSLYLKELKALSLWWRQLYDSVQLSYARDCLVESYFWTCAMFHGEDYSRARIIFAKVFQLMTMTDDIYDIHATLEECYKFNEAVQRWDKSAVSILPEYLRNFYIRILNDFDEMEDSLEPDEKHRMSNVKSSFKQQSEYYLREAQWSSDKHMPSFAEHLDVSFMSIGYPTMAVVVLLCARDGNGAAASMEASEWAPSLVRAGGEVTRFLNDIASYKTGKSGKDAASTIECYMAERGVGGEEAVAAVAALVESAWRTINRACVEMDPNLLPAARLLVNLATTPEVIYFGGRDGYTVGADLKGLVTALFLDPLPV

SEQ ID NO: 7 - protein, 677 aa, Oryza rufipogon
  REGION 1..677: note = putative protein
MAERRSTRRTSPALTVATGSPCHRRLLSRCLPPTPPLSPLANDASSPVPHRRLLSRAANAILCTPRLKTTMTSSGAMAAMESRRLGAEGQSRRRRVGGQRGRPRAPLPAPPRSNSPVATDASPCALANLHQRERERDLGRREEKWGTKCKYVATLDRHRSEEWMRERVDRLKMQVGCKILKTINVPYTVMLVDVLERLHIDNHFRDEIATALQHVFHHDEQQKAAAGFDDGDQLYLESLRFRLLRQHGFWVSADVFDKFKDSTGCFRESLSTDARGLLSLYNAAHLAMPGEAALDDAIAFSRRSLQSLQGALRSPMAKQVSRALDIPLPRAPKLLETMHYITEYEQEAAHDGMVLELARLDFELVRSLYLKELKALSLWWRQLYDSVQLSYARDCLVESYFWTCAMFHGEDYSRARIIFAKVFQLMTMTDDIYDIHATLEECYKFNEAVQRWDKSAVSILPEYLRNFYIRILNDFDEMEDSLEPDEKHRMSYVKSSFKQQSEYYLREAQWSSDKHMPSFAEHLDVSFMSIGYPTMAVVVLLCARDGDGAAASMEASEWAPSLVRAGGEVTRFLNDIASYKTGKSGKDAASTIECYMAERGVGGEEAVAAVAALVESAWRTINRACVEMDPNLLPAARLLVNLATTPEVIYFGGRDGYTVGADLKGLVTALFLDPLRV

SEQ ID NO: 8 - protein, 519 aa, Oryza sativa
  REGION 1..519: note = putative protein fragment
  source 1..519: note = Oryza sativa Japonica Group
RSEEWMRERVDRLKMQVGCKILKTINVPYTVMLVDVLERLHIDNHFRDEIATALQHVFHHDEQQKAAAGFDDGDQLYLESLRFRLLRQHGFWVSADVFDKFKDSTGCFRESLSTDARGLLSLYNAAHLAMPGEAALDDAIAFSRRSLQSLQGALRSPMAKQVSRALDIPLPRAPKLLETMHYITEYEQEAAHDGMVLELARLDFELVRSLYLKELKALSLWWRQLYDSVQLSYARDCLVESYFWTCAMFHGEDYSRARIIFAKVFQLMTMTDDIYDIHATLEECYKFNEAVQRWDKSAVSILPEYLRNFYIRILNDFDEMEDSLEPDEKHRMSYVKSSFKQQSEYYLREAQWSSDKHMPSFAEHLDVSFMSIGYPTMAVVVLLCARDGDGAAASMEASEWAPSLVRAGGEVTRFLNDIASYKTGKSGKDAASTIECYMAERGVGGEEAVAAVAALVESAWRTINRACVEMDPNLLPAARLLVNLATTPEVIYFGGRDGYTVGADLKGLVTALFLDPLRV

SEQ ID NO: 9 - protein, 600 aa, Oryza sativa
  REGION 1..600: note = putative protein
  source 1..600: note = Oryza sativa Japonica Group
MAAMESRRLGAEGQSRRRRVGGQRGRPHAPPAPPRSNSPVATDASPCALANLHQRERERDLGRQEEKWGTKCKYVATLDRHRSEEWMRERVDRLKMQVGCKILKTINVPYTVMLVDVLERLHIDNHFRDEIATALQHVFHHDEQQKAAAGFDDGDQLYLESLRFRLLRQHGFWVSADVFDKFKDSTGCFRESLSTDARGLLSLYNAAHLAMPGEAALDDAIAFSRRSLQSLQGALRSPMAKQVSRALDIPLPRAPKLLETMHYITEYEQEAAHDGMVLELARLDFELVRSLYLKELKALSLWWRQLYDSVQLSYARDCLVESYFWTCAMFHGEDYSRARIIFAKVFQLMTMTDDIYDIHATLEECYKFNEAVQRWDKSAVSILPEYLRNFYIRILNDFDEMEDSLEPDEKHRMSYVKSSFKQQSEYYLREAQWSSDKHMPSFAEHLDVSFMSIGYPTMAVVVLLCARDGDGAAASMEASEWAPSLVRAGGEVTRFLNDIASYKTGKSGKDAASTIECYMAERGVGGEEAVAAVAALVESAWRTINRACVEMDPNLLPAARLLVNLATTPEVIYFGGRDGYTVGADLKGLVTALFLDPLRV

SEQ ID NO: 10 - protein, 519 aa, Oryza glaberrima
  REGION 1..519: note = putative protein
RSEEWMRERVDQLKMQVRCKILKTTNVPYTVMLVDVLERLHIDNHFCDEIATALQHVFHHDEQEKADAGFDDGDQLHLESLRFRLLRQHGFWVYADVFDKFKDRTGCFRESLSTDARGLLSLYNAAHLAMPGEAALDDAIAFSRRSLQSLQGALRSPMAEQVSRALDIPLPRAPKLLETMHYITEYEQEEAYDGMVLELARLDFELVRSLYLKELKALSLWWRQLYDSVQLSYARDCLVESYFWTCAMFHGEDYSRARIIFAKVFQLMTMTDDIYDIHATLEECYKFNEAMQRWDKSAVSILPEYLRTFYIRILNDFDEMEDSLEPDEKHRMSYVKSSFKQQSEYYLREAQWSSDKHMPSFAEHLDVSFMSIGYPTMAVVVLLCARDGDGAAASMEASEWAPSLVRAGGEVTRFLNDIASYKTGKSGKDAASTIECYMAERGVGGEEAVAAVAALVESAWRTINRACVEMDPNLLPAARLLVNLATTPEVIYFGGRDGYTVGADLKGLVTALFLDPLPV

SEQ ID NO: 11 - protein, 505 aa, Oryza sativa
  REGION 1..505: note = putative protein
  source 1..505: note = Oryza sativa Japonica Group
MQVGCKILKTINVPYTVMLVDVLERLHIDNHFRDEIATALQHVFHHDEQQKAAAGFDDGDQLYLESLRFRLLRQHGFWVSADVFDKFKDSTGCFRESLSTDARGLLSLYNAAHLAMPGEAALDDAIAFSRRSLQSLQGALRSPMAKQVSRALDIPLPRAPKLLETMHYITEYEQEAAHDGMVLELARLDFELVRSLYLKELKALSLWWRQLYDSVQLSYARDCLVESYFWTCAMFHGEDYSRARIIFAKVFQLMTMTDDIYDIHATLEECYKFNEAVQRWDKSAVSILPEYLRNFYIRILNDFDEMEDSLEPDEKHRMSYVKSSFKQQSEYYLREAQWSSDKHMPSFAEHLDVSFMSIGYPTMAVVVLLCARDGDGAAASMEASEWAPSLVRAGGEVTRFLNDIASYKTGKSGKDAASTIECYMAERGVGGEEAVAAVAALVESAWRTINRACVEMDPNLLPAARLLVNLATTPEVIYFGGRDGYTVGADLKGLVTALFLDPLRV

SEQ ID NO: 12 - protein, 505 aa, Oryza nivara
  REGION 1..505: note = putative protein
MQVGCKILKTINVPYTVMLVDVLERLHIDNHFRDEIATALQHVFQHDEQEKAAAGFDDGDQLHLQSLRFRLLRQHGFWVSADVFDKFKDSTGCFRESLSTDARGLLSLYNAAHLAMPGEAALDDAIAFSRRSLQSLQGALRSPMAKQVSRALDIPLPRAPKLLETMHYITEYEQEEAHDGMVLELARLDFELVRSLYLKELKALSLWWRQLYDSVQLSYARDCLVESYFWTCAMFHGEDYSRARIIFAKVFQLMTMTDDIYDIHATLEECYKFNEAVQRWDKSAVSILPEYLRNFYIRILNDFDEMEDSLEPDEKHRMSYVKSSFKQQSEYYLREAQWSSDKHMPSFAEHLDVSFMSIGYPTMAVVVLLCARDGNGAAASMEASEWAPSLVRAGGEVTRFLNDIASYKTGKSGKDAASTIECYMAERGVGGEEAVAAVAALVESAWRTINRACVEMDPNLLPAARLLVNLATTPEVIYFGGRDGYTVGADLKGLVTTLFLDPLPV

SEQ ID NO: 13 - protein, 514 aa, Oryza barthii
  REGION 1..514: note = putative protein
  UNSURE 498: note = X may be any amino acid
MRERVDQLKMQVRCKILKTTNVPYTVMLVDVLERLHIDNHFCDEIATALQHVFHHDEQEKADAGFDDGDQLHLESLRFRLLRQHGFWVYADVFDKFKDRTGCFRESLSTDARGLLSLYNAAHLAMPGEAALDDAIAFSRRSLQSLQGALRSPMAEQVSRALDIPLPRAPKLLETMHYITEYEQEEAYDGMVLELARLDFELVRSLYLKELKALSLWWRQLYDSVQLSYARDCLVESYFWTCAMFHGEDYSRARIIFAKVFQLMTMTDDIYDIHATLEECYKFNEAMQRWDKSAVSILPEYLRTFYIRILNDFDEMEDSLEPDEKHRMSYVKSSFKQQSEYYLREAQWSSDKHMPSFAEHLDVSFMSIGYPTMAVVVLLCARDGDGAAASMEASEWAPSLVRAGGEVTRFLNDIASYKTGKSGKDAASTIECYMAERGVGGEEAVAAVAALVESAWRTINRACVEMDPNLLPAARLLVNLATTPEVIYFGGRDGYTVGXDLKGLVTALFLDPLPV

SEQ ID NO: 14 - protein, 466 aa, Oryza sativa
  REGION 1..466: note = putative protein
  source 1..466: note = Oryza sativa Japonica Group
MQVGCKILKTINVPYTVMLVDVLERLHIDNHFRDEIATALQHVFHHDEQQKAAAGFDDGDQLYLESLRFRLLRQHGFWVSADVFDKFKDSTGCFRESLSTDARGLLSLYNAAHLAMPGEAALDDAIAFSRRSLQSLQGALRSPMAKQVSRALDIPLPRAPKLLETMHYITEYEQEAAHDGMVLELARLDFELVRSLYLKELKALSLWWRQLYDSVQLSYARDCLVESYFWTCAMFHGEDYSRARIIFAKVFQLMTMTDDIYDIHATLEECYKFNEAVQRWDKSAVSILPEYLRNFYIRILNDFDEMEDSLEPDEKHRMSYVKSSFKQQSEYYLREAQWSSDKHMPSFAEHLDVSFMSIGYPTMAVVVLLPGKSGKDAASTIECYMAERGVGGEEAVAAVAALVESAWRTINRACVEMDPNLLPAARLLVNLATTPEVIYFGGRDGYTVGADLKGLVTALFLDPLRV

SEQ ID NO: 15 - protein, 502 aa, Oryza rufipogon
  REGION 1..502: note = putative protein
  UNSURE 464: note = X may can be any amino acid
MAYTVMLVDVLERLHIDDHFRDEITTALQHVLLHHEEQKSVVTVDQLHLESLRFRLLRQHGFWVSADVFDKFKDNTGCFKESLSTDARGLLSLYNAAHLAMPGETALDNAIAFSRRSLQSLQGKLRSPMAEQVSRALDIPLPRTPKLLETMRYITEYEQEEAHDSVVLDLARLDFELIRSLYLKELKTLSLWWTELYDSIKLSYARDRIVESYFWTCAMFHGEDYSRARIIFVKVFQLVIMMDDTYDIHATLEECHKFNEAMQRWDKSAVCILPEYLRLFYIKMLNNFDEMKDSLEPDEKYRMSYVKTSLKQLSEYFLREAQWSSDKHMPSFAEHLDVSCMSSGYPTMALVAVLLCVRDGDGVAASMEASEWTPSLVRAGGEVTRFLNDVASYKTGKSGKDGASTIECYMAERGVGGEEAVAAVAALVESAWRTINRACVEMDPNLLPAARLLVNLATTPEVIXPEVIYFGGRDGYTVGADLKGLVTALFLDPLPVNQPQQS

SEQ ID NO: 16 - protein, 504 aa, Oryza meridionalis
  REGION 1..504: note = putative protein
MAYTVMLVDVLERLHIDNHFRDEIATALQHVLLHHEEHADSVAAVDLLHLESLRFRLLRQHGFWVPADVFDKFKDSTGCFRESLSTDARGLLSLYNAAHLAMPGEEAALNDAIAFSRRSLQSLVQGKLGSPMAEQVSRALDIPLPRTPKLLETMRYITEYEQEEAHDGVVLELARLDFKLIRSLYLNELKTLSLWWRQLYGSVKLSYARDRLVESYFWSCAIFHGEKYSRSRIIFTKVFQLMTLVDDTYDIHATLKECYKLNEAMQRWDKSAVSILPEYLRVFYIKLLNDFDEMEDSLEPDEKYRMSYAKTTFKQMSEYYLREAQWSSDKYMPSFAEHLDISLMSSGFPAMAPVLLLGVRDRGSCGGAAAATKEAFEWATSVPVPALVRAGGELARFLNDVASYRIGKSGGDMASTVECYMAERGVNGDEAVAAVAALAERAWRTINGECVAGLEPALLPAAWLMVNLARTVEVIYLGGRDGYTVGGDLKGLVSNLFLDPLPLY

SEQ ID NO: 17 - protein, 428 aa, Oryza barthii
  REGION 1..428: note = putative protein
MALMDLITRAYRSEKWSREQVDHLKRQVHCKLLKATSMAYTVMLVDVLERLHIDDHFRDEITTVLQHVLLHHEEQKSVVTVDQLHLESLRFRLLRQHGFWVSADVFDKFKDNTGCFKESLSTDARGLLSLYNAAHLAMPGETALDNAIAFSRRSLQSLQGKLRSPMAEQVSRALDIPLPRTPKLLETMRYITEYEQEEAHDSVVLDLARLDFELIRSLYLKELKTLSLWWTELYDSIKLSYARDRIVESYFWTCAMFHGEDYSRARIIFVKVFQLVIMMDDTYDIHATLEECHKFNEAMQRWDKSAVCILPEYLCLFYIKMLNNFDEMKDSLEPDEKYRMSYVKTSLKQLSEYFLREAQWSSDKHMPSFAEHLDVSCMSSGYPTMALVAVLLCVRDGDGVAASMEASEWTASLVRAGGEVTRFLNDKK

SEQ ID NO: 18 - protein, 947 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTGDDDDKLIMSGSKVISVLDTKVLAVKGGTMTQPVAAATTGRACSPSLWGDFFVTYIPPKPQRSEEWMRERVDWLKMQVGCKILKTINVPYTVMLVDVLERLHIDNHFRDEIATALQHVFHHDEQQKAAAGFDDGDQLHLESLRFRLLRQHGFWVSADVFDKFKDSTGCFRESLSTDARGLLSLYNAAHLAMPGEAALDDAIAFSRRSLQSLQGALRSPMAEQVSRALDIPLPRAPKLLETMHYITEYEQEAAHDGMVLELARLDFELVRSLYLKELKALSLWWRQLYGSVQLSYARDCLVESYFWTCAMFHGEDYSRARIIFAKVFQLMTMTDDIYDIHATLEECYKFNKAIQRWDKSAVSILPEYLRNFYIRILNDFDEMEDSLEPDEKHRMSYVKSSFKQQSEYYLREAQWSSDKHMPSFAEHLDVSSMSIGYPTMAVVVLLCARDGDGAAASMEASEWAPSLVRAGGEVTRFLNDIASYKTGKSGKDAASTIECYMAERGVGGEEAVAAVAALVESAWRTINRACVEMDPNLLPAARLLVNLATTPEVIYFGGRDGYTVGADLKGLVTALFLDPLRV

SEQ ID NO: 19 - DNA, 2844 bp, synthetic construct
  source 1..2844: mol_type = other DNA
atgaagatcgaggaaggcaagctcgtgatctggatcaacggcgacaagggctacaacggcctggccgaggtgggcaagaagttcgagaaggacaccggcatcaaggtgacggtggagcacccggacaagctcgaggagaagttcccgcaggtggcggccacgggcgacggcccggacatcatcttctgggcccatgaccgcttcggcggctacgcccagtcgggcctgctggccgagatcaccccggacaaggcgttccaggacaagctctatcccttcacgtgggacgccgtgcgctacaacggcaagctgatcgcgtatcccatcgcggtggaggccctgtcgctcatctataacaaggacctgctcccgaacccgcccaagacctgggaggagatccccgccctcgacaaggagctgaaggccaagggcaagtcggcgctcatgttcaacctgcaggagccgtacttcacctggcccctgatcgcggccgacggcggctacgcgttcaagtatgagaacggcaagtatgacatcaaggacgtgggcgtggacaacgcgggcgccaaggccggcctgaccttcctcgtggacctgatcaagaacaagcacatgaacgccgacacggactactcgatcgcggaggccgcgttcaacaagggcgagaccgccatgacgatcaacggcccgtgggcgtggtcgaacatcgacacctcgaaggtgaactatggcgtgaccgtgctccccacgttcaagggccagccctcgaagcccttcgtgggcgtgctgtcggcgggcatcaacgccgcgtcgccgaacaaggagctcgcgaaggagttcctggagaactacctgctcaccgacgagggcctggaggccgtgaacaaggacaagcccctgggcgccgtggccctgaagtcgtatgaggaagagctggtgaaggacccgcgcatcgcggccaccatggagaacgcgcagaagggcgagatcatgccgaacatcccccagatgtcggccttctggtatgcggtgcgcaccgccgtgatcaacgcggcctcgggccgccagaccgtggacgaggccctcaaggacgcccagaccggcgacgacgacgacaagcttatcatgtcgggctcgaaggtgatctcggtcctcgacaccaaggtcctggctgtcaagggcggcacgatgacgcagccggtggccgcagcgaccacggggcgcgcctgctcgcccagcctctggggcgatttcttcgtgacctatatcccgccgaagccgcagcggtcggaagaatggatgcgcgagcgggtggactggctgaagatgcaggtgggctgcaagatcctgaagacaatcaacgtgccctataccgtgatgctggtggacgtgctggagcgtctgcatatcgacaaccatttccgcgacgagatcgccaccgccctgcagcatgtgttccatcacgacgagcagcagaaggccgcagctggcttcgatgacggcgaccagctgcaccttgagagcctgcgctttcggctcctgcggcagcacggcttctgggtgtcggccgacgtcttcgacaagttcaaggacagcaccggctgcttccgcgagtcgctgtcgaccgatgcccgggggctgctcagtctctacaacgccgcgcacctcgccatgccgggcgaggccgcccttgacgatgcgatcgccttctcgcggcgctcccttcagtcgctgcagggcgcgctgcgcagcccgatggccgagcaggtgtcgcgcgccttggatattccgctgccgcgcgcgcccaagctcctggagacgatgcactacatcaccgagtacgagcaggaggcggcccatgacggcatggtgctcgaactcgcgcgcctcgacttcgagctcgttcggtcgctctatctcaaggagctgaaggcgctctcgctctggtggcggcaattgtacggctccgtgcagctcagctatgcccgcgactgcctcgttgagagctacttctggacctgcgcgatgttccacggggaggactattcgcgcgcgcggatcatcttcgccaaggtcttccagctgatgaccatgaccgacgatatctacgacatccacgcgacgctcgaggaatgctacaagttcaacaaggccatccagcgctgggacaagtcggccgtgtcgatcctccccgaatacttacgcaacttctacatccgcatcctcaatgacttcgatgagatggaggatagccttgagcccgacgagaagcatcggatgtcctatgtcaaatcctcgttcaagcagcagagcgaatattacctgagggaggcgcagtggagctccgacaagcacatgccctcgttcgcggagcatctggatgtgtctagcatgagcatcggctatcccacgatggcggtggtggtcctgctctgcgcccgcgatggagacggggcggccgcctcgatggaagcgagcgagtgggcgccgtcgctggtgcgggcgggcggtgaggtgacacgcttcctgaacgacatcgcgagctacaagaccggcaaatccgggaaggacgcggcgtccacgatcgagtgttacatggccgagcgcggcgtgggcggcgaggaggccgtcgccgcggtggcggcgctcgtggagtcggcctggcgcaccatcaatcgggcctgcgtcgagatggacccgaacctcctgcccgcggcccgactgctggtgaacctggcgacgacgcctgaggtcatctatttcggcggccgcgacgggtatacggtcggcgccgatctgaaagggctcgtcaccgcactgtttctcgacccgctccgggtgtga

SEQ ID NO: 20 - protein, 9 aa, synthetic construct
  VARIANT 3: note = X may be any amino acid, preferably A, S, M or P
  VARIANT 5..8: note = X may be any amino acid
GRXCXXXXW

SEQ ID NO: 21 - protein, 572 aa, synthetic construct
MSGSRVISVLDTKVLAVKAGSMTQPVAAATTGRACSPSLWGDFFVTYIPPKPQRSEDWMRERIDWLKMQVGCKILKTINVPWTVMLVDVLERLHIDNHFRDEIATGLNHVFHHEEQNKAAAGFDDADQLHLESLRFRLLRQHGFWVSADVFDKFKDSTGCFKDSLSTDAKGVLSLYNAAHLAMPGEAALDDALAFSRRSLQTVQGALRSPMAEQVSMALDIPIPRAPKLLETMHWITEYDQEAAHDGMVLELARLDFELVRSVYLKELMGLSLWWRQIYGSLQLSYARDCLVESYWWTCAMFHGEDYSRARIIFGKVFQLMTMTDDIYDIHATLEECYKFNKGIQRWDRSAVSILPEYLRQFYVRIVNDFDEMEDSLEPDEKHMMSYVKSSFMQQSEYYLREAQWSSDRHMPSYAEHVDVSTMSIGYPTMGIVVLLCGRDADGAAASMEASEWGPSLIRGGGEVTRFVNDIASYKTGKSGKDAASTIECYMAERGVGGDEAVAAVAALVESGWRTINKACIEMDPQLVPAARLLVNIATTPEVIYFGGRDGYTLGADLKGLVTAIFIDPLRV

SEQ ID NO: 22 - protein, 572 aa, synthetic construct
MSGSKVITVLDTKVLAVKGASMTQPVAAATTGRACSPSLWADFYVTYIPPKPQRSEEWKREKLDWLKMQVGCKIIKTINVPYTVKLVDVLERLHIDNHFRDEIATALNHLYHHDEQNMAAAGFDDGDQLHLESLRFRLLRQHGFWVSADVFDKFKDSTGCFMESLSTDGRGLLSLYNAAHLAMPGEAALDDAIAFSRRSLQSLQGAVRSPMAEQVSMALDIPLPRAPKLLETMHYITEYEQEAAHDGMVLELARLDFELLRSLYLKELRAISVWWRQLYGSVQLSYARDCLVESYFWTCAKFHGEDFSRARIIFAKLFQLMTKTDDVYDLHATLEECYKFNRAVQRWDMSAVSILPEYLRNFYIRILNEFDEREDTLEPEEKHMMSYVKSSFMQQSEWFLREAQWSSDKHMPSFGEHLDLSSMSIGYPTMAVLVVLCARDADGAGASMEASEWAPSLIRAGGEVTRFLNDIASYKTGKSGKDAASTIECYMAERGLGGEEALAAVAALLESAWRTVNRACVEMDPNILPAARLVVNLATTPEVIYFGGRDGYTLAADLRGLVTALWLDPLRL

SEQ ID NO: 23 - protein, 572 aa, synthetic construct
MSGSKVISLVETKVVAVKGGTRTQPVAGGTTGRACSPSIWGDFFVTFIPPKPNRTDEWRRERVEWLKKQVGCRILRSINVPYTVMLVDLLERLHIDNHFRDEIASALQHVFHHEEQQKAAAGFDDGDQLHLESLRFRLLRQHGFWVSADVFDKFKDTTGCFKETLTTDARGLLSLYNAAHLAKPGEAALDDAVGFSRRSLQSVQGGLMSPRGDQVSMALDLPLPRAPKLIETMHFITEYEQEAAHDGMVLELARIDFELVRSLYLKELKAISLWWRQIYGSIQLSYARDCLVETYFYTCAMFHAEDYTRARIIFAKVFQLKTMTDDLYDIHATLEECYKFNKAIQRWDKSAVSILPEFLRNFYVMVLNDFDEMDDSLEPEEKHRRSYVMSSFKQQSDYFLREAQWSSDKHMPSFAEHLDISSMSIGFPTMGIVLLLCGRDAEGGAASMEASEWAPSLIRAGGEVTRFLNDIASYKTGKSGKEAASSIECYMAERGVGGEEALAAVGAVVESAWRTLNMACIEMDPNLLPAARILVNLATTPEVVYYGGRDGYTVGAEIMGIVTGLFLEPLMV

SEQ ID NO: 24 - protein, 572 aa, synthetic construct
MSASKLISLIDTMILGVKGGTMTQPVGGATSGRACSPTLWGEFFVTFVPPKPQRSEEFMMERVDWLKMQVGCKILKTINVPWTVMLVDVLERLHIDNHFREEIATALQHVFHHEEQNKAAAAFDDGDQLHLESLRFRLLRQHGYWISAEVFDKFKDSTGCFKESLSSDAKGLLSLYNAAHLAMPGEAGLDDAIAFSRRSLNTLQGALRSPMADQVSKALDVPLPRAPMLLESKHYITEYEQEAAHDGKVLELAMLDFEVVRSLYIKELKALSLWWRQLYASVQLSYGRECLVESYFYTCAMFHGEDYSRGRLIFAKVFQLMTMTDDIYDLHATVEECYKFNKALNRWDKSAVSILPDWLRNFYIRILNDYDEMEDTLDPDEKHKMSYLKSSFMQQSEYFLREANWSSDRHMPSFAEHIDVSSMSLGFPTRAVVVLLCGRDGDGAAATRDASEWAPTLVRAGGEVSRFLNDIASWKTGKSGMDAASTIECYMAERGVAGDEAVAAVAALVESAWRTINMACVEMEPNLIPGGRLLLNLATSPEVIYFGGRDGYTVGADLKGLVTAVFVEPLRI

SEQ ID NO: 25 - protein, 572 aa, synthetic construct
MSGSKVLSLLDTKVLAVMGGSMTNPIGAGTSGRACSPSLWGDFFVTWVPPKPQMSEDFMMERIEWLKRNVACMLLKTINIPYTVMVVDVLDRLHIDNHFRDEIGTGLNHVFHHDENQRAGGGFDDADQLHLESLKFRLLRNHGFWISADVFDRFKDSTGCFRDTLSTDARGVLTLYNAAHIGMPGEAALDDAIGFSKRSLQSVQGAIKTPMADQVSRALDVPLPRAPKLLETRHWITEYDQEGAHDGRVLELARIDFEIVRSLYIKELRALSLWWRQLYGSVQLSYARDCILESYFWTCAMYHGEEYSRGRLIWAKVFNVMTMTDDVYDIHATIEECYKFNRAIQRWDKSAISVLPEYLRNFYIRILNDFDDMEESLDPDEKHRRSYVRSSWKQQSEYYLKEAQWSSDKHMPSFAEHLDVSSMSIGYPTRAVIILLCARDADGAGATKEATEWAPSLVRGGGEVTRFVNDIASYRTGRSAKEAATTIECYMGERGVGGEEAVGAIAAIVESAWRTINRACVEREPNLLPAAMLLVNLGTTPELIYWAGRDGFTVAGDLRGIVTAIFIDPLRI

SEQ ID NO: 26 - protein, 572 aa, synthetic construct
MSGSKVVTVIETKVLAVKGASMSQPVGAATTGRACSPSLWGDYFVSWLPPKPQRSDEFKRERVDYLKMNVGCKILMSIQVPYTVMLVDVLERLHIENHFRDEIGTALQHLWHHEEQQKGGAGYDDGDNLHVESLMFRLLRNHGFWVSADLFDKFKDTTGCFMESLSSDAKGLLTLYNAAHVAMPGEAALDDAIAFSKRSLQSINGAIMSPKAEQVSRALDIPLPMAPMLLESMHYITEYEQDGGHDGKVLDLARVDFELLRSLYLMELRALTVWWRQLYATIQLSYARDCLVESYFWTCAMFHGEDFTRARLIFAKVWQLMTRTDDLYDLHATLEECYKFNKALQRWDKTGLSVLPDYLRNFYIRIVQEFDEKDESLDPDEMHRMSWLKSTYRQQSEWYLKEAQWSSEKHKPSFAEHLDISSMSVGFPTRAVIVVLCGRDGDGAGASREGTEWAPSLLRGGAEVTRFLNDVASFKTGRSGREAASSIDCYMGERGVAGEEAVGAVAAVIETGWRTVNRACIEMDPQLVPAARILVNIGTTPELIYYAARDGYTLAAEIMGVLSALWIEPIRV

SEQ ID NO: 27 - protein, 572 aa, synthetic construct
MSGSKIVSVIDSKLLAIKGGSKTNPLGAGTSGRACTPSIWGDWFLTWIPPRPQMSEEYMKEMVEYLKMQVGCRIIKTINIPYTVMLVDLLERLHIEQHFKEEIASALNHIWHHDEQNRAAGAWDEGDNLHLESLRFRLLRQHGFYISAELFEKFKDSTGCFRESVSTDAMGLLSLYNAAHLGMPGEAALDEALGFSRRSLQTLQGGLKSPMGDQVSRALEIPVPRAPMLVDTRHFITEYEQEAGHDAMVLDLAKLDFEIIRSLYLMELKAISVWWMQIYASLQLTYGRECLLETYWFTCARWHGEEFTMARIIFAKLFQLMSMSDDIYDVHATIEECYKFNRAIQRWDKTGISVLPEWLRNYYLKIVNEFDDMEESLDPEDRHKMTYIKSSFKQQTEYFLREAQWSSDKHKPSFGEHLELSSMSVAFPTRAVLIVVCARDGDGAAATMEASEFAPSIVKAAGEVSRFLNDVATYRTGKSGKDGASTIECYMGEMGVGGDDALGGIAAVVESAWRSINRGCVEMDPNVIPAGMILLNVATSPDIVYFGGMDGFTVGAELMGVVTAVFLEPLKV

SEQ ID NO: 28 - protein, 572 aa, synthetic construct
MTGSRVISVVDTKVLAVKGGTMSQPVGAASSAKGCTPTLWAEYWLTWLPPKPQRSEEYKRDMLDYLKRNVGCKVIKTVNIPWSVMIVDVLERLHIENHFMDEIGSALQHLWHHDENNKAGGGFDDGDQLHLESLRFRLLRQHGFYVSGDIFDKFRDTTGCFRETITTDAKGLLTLYNAAHLGKPGEGGLDDAIAFTKRTLQSLQGAVKSPMADQVTMGLELPLPRAPRLLESMHYITEYDQEAGHEGMVLELAMIDFELVRSLYVKELMGVSLWWKQLFGSLQLSYARDCLIESYYWSCAKFHGEDYSKARVVWGKLYNIKTMSDDVYDVHATIEECFRFNKGLNRWDMSAVSILPDYLRQYYIRVINEFDEKDDTLEPDEKHKRSYIMSTWKQQTDFFIMEANWSSDKHMPSWAEHIEITTMSVAFPTRALLLILCAKEADAAGGSRDGSEWAPSIIRGAGDVTRFLNDVATYMTGRSAREAGTTIECYMAERGLGGEEAVAGVAAVLDTAWRSLNMACVDRDPQILPGARILVNLATTPELLYYGAMDAFTLAGELRAILSAVWLDPIMV